(19) United States
(12) Patent Application Publication
(10) Pub. No.: US 2025/0143627 A1
(43) Pub. Date: May 8, 2025

(54) METHODS AND SYSTEMS FOR MULTI-CLASS CLASSIFICATION OF SEIZURE

(71) Applicant: Ceribell, Inc., Sunnyvale, CA (US)

(72) Inventors: Archit GUPTA, Sunnyvale, CA (US); Baharan KAMOUSI, Redwood City, CA (US); Suganya KARUNAKARAN, Sunnyvale, CA (US)

(21) Appl. No.: 18/769,039

(22) Filed: Jul. 10, 2024

Related U.S. Application Data
(60) Provisional application No. 63/513,317, filed on Jul. 12, 2023.

(51) Int. Cl. A61B 5/00; A61B 5/372; G16H 10/60; G16H 50/20

(52) U.S. Cl. CPC A61B 5/4094; A61B 5/372; A61B 5/725; A61B 5/7267; G16H 10/60; G16H 50/20

(57) ABSTRACT

Described herein are methods and systems for the classification of seizure in a subject. The systems may include a data module configured to obtain a plurality of electroencephalography (EEG) signals collected from a subject. The systems may also include a processing module in communication with the data module. The processing module may be configured to process the data to detect and monitor seizures or related symptoms that the subject is experienced or is experiencing. The processing module may also generate indications or assessments for seizure at an individual level.

### CROSS-REFERENCE TO RELATED APPLICATIONS

This application claims priority to U.S. Provisional Application No. 63/513,317, filed on Jul. 12, 2023, which is hereby incorporated by reference in its entirety.

### FIELD

This application relates to methods and systems for the classification of seizure in a subject.

### BACKGROUND

Seizures are neurological events characterized by abnormal electrical activity in the brain. Seizures may manifest with various intensities, may affect various regions of the brain (e.g., generalized seizures affect both sides of the brain, focal seizures affect one area of the brain) and may be associated with various conditions like epilepsy, brain tumors, stroke, and head injury. Thus, medical professionals must consider myriad factors when diagnosing and treating a patient experiencing seizures. Accordingly, accurate classification of seizures and abnormal brain activity related to seizures is paramount for diagnosing and effectively treating a patient experiencing seizures or related conditions.

Conventional methods for seizure classification may include using predictive classifiers to discriminate between electrographic seizure and non-electrographic seizure states based on electroencephalogram (EEG) signals recorded from a subject. Electrographic seizures refer to the presence of abnormal electrical discharges in the brain, but these seizures may or may not manifest as clinical symptoms. In contrast, highly pathological EEGs with a high likelihood of epileptiform activity may indicate abnormal EEG patterns (e.g., epileptiform discharges, sharp waves, or spikes) that strongly suggest the presence of an underlying seizure disorder, even in the absence of overt clinical manifestations. Thus, discriminating between electrographic seizures, epileptiform activity, and normal electrographic activity may improve predictions of future seizures, assessments of treatment efficacy, and monitoring the progression of underlying conditions. However, current methods lack classifiers for highly pathological EEGs with a high likelihood of epileptiform activity.

Further, current methods for seizure classification are often limited by the expert knowledge and experience required to interpret EEG data. The process may be time-consuming and subjective, leading to interobserver variability. Moreover, reliance on visual analysis of EEG recordings may result in missing or misclassified seizures, especially when detecting subtle or atypical seizure patterns. Another challenge for using conventional seizure classification methods relates to the inability to detect of seizures in real-time, which requires continuous monitoring and immediate recognition of abnormal electrographic activity.

Accordingly, it would be beneficial to have alternative methods and systems for seizure classification.

### SUMMARY

Described herein are methods and systems for classifying seizures or related activity (e.g., epileptiform activity) in a subject. The classification of seizure may be based on output from one or more predictive models (e.g., machine learning models) that process a plurality of features extracted from electroencephalography (EEG) signals recorded from a subject. The output of the one or more prediction modules may be, or may be used to generate, an overall seizure classification of the subject's brain activity over a period of time.

The method for classifying seizures may include: obtaining data comprising electroencephalography (EEG) signals recorded from a subject over a plurality of channels; pre-processing the data by: dividing the data into a plurality of temporal segments, and extracting a plurality of features from each of the temporal segments; generating a preliminary seizure classification for each of the temporal segments based on the plurality of extracted features; and determining an overall seizure classification for the subject based on the preliminary seizure classification for each of the temporal segments.

In some variations, the overall seizure classification may be a ternary classification of electrographic seizure-like activity, highly pathological EEG with high likelihood of epileptiform activity, or normal electrographic activity. In other variations, the overall seizure classification may be a seizure probability or a seizure severity value.

The temporal segments may be continuous temporal segments. In some variations, each of the temporal segments may be about 10 seconds. The plurality of features may include at least one time-domain feature. Alternatively, the plurality of features may include at least one frequency-domain feature.

The EEG signals may be recorded from a plurality of electrodes, e.g., electrodes incorporated into a headband worn by the subject. The EEG signals may be obtained and recorded from a plurality of channels. In some variations, the number of channels may include 8 channels. In some variations, the method may include treating the subject for seizure if one or both of electrographic seizure and highly pathological EEG with high likelihood of epileptiform activity and is detected.

In some variations, generating the preliminary seizure classification may include generating a first probability of seizure under a first comparison and generating a second probability of seizure under a second comparison. Generating the first probability of seizure may include comparing a first likelihood of a temporal segment as normal electrographic activity to a second likelihood of the temporal segment as highly pathological EEG with high likelihood of epileptiform activity. Determining the overall seizure classification may then include calculating a moving average of the first probability of seizure over the time window. In some variations, generating the second probability of seizure may include comparing a first likelihood of a temporal segment as electrographic seizure to a second likelihood of the temporal segment as highly pathological EEG with high likelihood of epileptiform activity. Generating the first and second probabilities of seizures may then include filtering the each of the temporal segments with cascaded convolutional filters. In some variations, generating the preliminary seizure classification may include combining the first and second seizure probabilities and classifying a corresponding temporal segment based on the combined seizure probabilities.

Determining the overall seizure classification may include determining the overall seizure classification for a time window. In some variations, the time window may include about 3 temporal segments. In some variations, determining the overall seizure classification may include calculating a moving average of the preliminary seizure classification over the time window. Each temporal segment may correspond to at least one epoch, and the time window may include one or more epochs. In some variations, pre-processing the data may include extracting a plurality of multi-channel features that quantify a degree of correlation between pairs of temporal segments from different EEG signals corresponding to a given time epoch. In some variations, the method may include using a multichannel machine learning model to generate a multi-channel seizure classification for each time epoch based on the plurality of multi-channel features. The time window may have a duration that encompasses one time epoch. In some variations, the time window may have a duration that encompasses a plurality of successive time epochs. The plurality of successive time epochs may be non-overlapping. In some variations, the plurality of successive time epochs may overlap by 50% or less. The duration of each of the time epochs may range from about 1 second to about 10 minutes. For example, the duration of each of the time epochs may be about 10 seconds, about 30 seconds, about 60 seconds, about 2 minutes, about 5 minutes, or about 10 minutes.

In some variations, the method may include providing a trace of the overall seizure classification over time. In other variations, the method may include determining a trendline of the trace.

In some variations, the plurality of features may include at least one feature that quantifies a degree of correlation of at least one of the plurality of temporal segments with a corresponding time-based segment of at least one other simultaneously collected EEG signal. In some variations, an EEG signal from the at least one of the plurality of temporal segments and the at least one other simultaneously collected EEG signal may be collected from the same hemisphere of a brain.

Systems for detecting seizure are also described herein. An exemplary system may include: a data module configured to receive data having a plurality of electroencephalography (EEG) signals recorded during a time window and over a plurality of channels from a subject; and a seizure detection module having a memory storing a set of instructions and one or more processors that are configured to, responsive to the set of instructions: pre-process the data received by the data module by: dividing the EEG signal into a plurality of temporal segments, and extracting a plurality of features from each of the plurality of temporal segments; generate a preliminary seizure classification for each of the temporal segments based on the plurality of extracted features; and determine an overall seizure classification for the subject based on the preliminary seizure classification for each of the temporal segments.

In some variations, the overall seizure classification may be a ternary classification of electrographic seizure-like activity, highly pathological EEG with high likelihood of epileptiform activity, or normal electrographic activity. In other variations, the overall seizure classification may be a seizure probability or a seizure severity value.

The preliminary seizure classification may include a first probability of seizure under a first comparison and a second probability of seizure under a second comparison. The first probability of seizure may include a comparison of a first likelihood of a temporal segment as normal electrographic activity to a second likelihood of the temporal segment as highly pathological EEG with high likelihood of epileptiform activity. The second probability of seizure may include a comparison of a first likelihood of a temporal segment as electrographic seizure to a second likelihood of the temporal segment as highly pathological EEG with high likelihood of epileptiform activity.

As previously described, the EEG signals may be obtained and recorded from a plurality of channels. In some variations, the plurality of channels may include 8 channels. In some variations, the system may include a headband and the headband may include a plurality of electrodes from which the plurality of electroencephalography (EEG) signals is recorded. The temporal segments may be continuous temporal segments. In some variations, each of the temporal segments may be about 10 seconds. In some variations, the plurality of features may include at least one time-domain feature. In other variations, the plurality of features may include at least one frequency-domain feature. The plurality of features may include at least one feature that quantifies a degree of correlation of at least one of the plurality of temporal segments with a corresponding time-based segment of at least one other simultaneously collected EEG signal.

The seizure detection module may determine the overall seizure classification by determining the overall seizure classification for a time window. In some variations, the time window may include about 3 temporal segments. In some variations, the overall seizure classification may include calculating a moving average of the preliminary seizure classification over the time window. Each temporal segment may correspond to at least one epoch, and the time window may include one or more epochs. In some variations, the time window may have a duration that encompasses one time epoch. In some variations, the time window may have a duration that encompasses a plurality of successive time epochs. In other variations, the plurality of successive time epochs may be non-overlapping. In some variations, the plurality of successive time epochs may overlap by 50% or less. The duration of each of the time epochs may range from about 1 second to about 10 minutes. For example, the duration of each of the time epochs may be about 10 seconds, about 30 seconds, about 60 seconds, about 2 minutes, about 5 minutes, or about 10 minutes.

### BRIEF DESCRIPTION OF THE DRAWINGS

FIG. 1 illustrates a schematic of a neurological condition detection and monitoring system, according to embodiments herein.

FIG. 2 illustrates a schematic of the various modules for a neurological condition detection and monitoring system, according to embodiments herein.

FIG. 3 illustrates a schematic of an interface of a neurological condition detection and monitoring system, according to embodiments herein.

FIG. 4 shows a schematic of a computer system that is programmed or otherwise configured to implement the methods described herein.

FIG. 5 shows a schematic of a seizure classification module in accordance with embodiments herein.

FIG. 6 shows a method of seizure classification in accordance with embodiments herein.

FIG. 7 shows an alternative method of seizure classification in accordance with embodiments herein.

### DETAILED DESCRIPTION

This application relates to methods and systems for the classification of seizure or related activity (e.g., epileptiform activity) in a subject. The classification of seizure may be based on output from one or more predictive models (e.g., machine learning models) that process a plurality of features extracted from electroencephalography (EEG) signals recorded from a subject. The output of the one or more prediction modules may be, or may be used to generate, an overall seizure classification of the subject's brain activity over a period of time. Any suitable therapy may be given to treat the subject (e.g., a drug, controlling the environment, addressing an underlying medical condition, etc.) based on the overall seizure classification.

### Seizure

Seizure is a neurological phenomenon characterized by abnormal, excessive electrical activity in the brain. It presents a range of clinical issues that may have significant implications for individuals and their overall health. Seizure occurrences may be unpredictable, disrupting daily activities and social interactions. Those living with seizures often experience apprehension about having a seizure in public or compromising their safety while engaging in tasks such as driving or operating machinery. Moreover, the consequences of seizures extend beyond the immediate event, leading to various physical and psychological complications. Physical injuries are common during seizures due to sudden loss of muscle control, resulting in falls, head injuries, and potential burns. Additionally, seizures may lead to emotional distress, anxiety, and a decreased quality of life for individuals and their families.

A lack of expert awareness and understanding of seizures among healthcare professionals may result in delayed diagnosis and treatment of seizure patients, which may have detrimental effects on patient outcomes. For example, research shows that up to one-third of newly diagnosed individuals with epilepsy in the United States remain untreated up to three years after diagnosis. To improve early detection and management, it is crucial for healthcare providers to be vigilant about the signs and symptoms of seizures. Several clinical assessment tools have been developed to aid in the identification and evaluation of seizures. These tools aim to provide a comprehensive understanding of seizure severity and associated factors. However, integrating these assessment protocols into routine clinical practice may be challenging. Passive methods that do not solely rely on behavior-based assessments administered by specialists are sought after in the ICU setting.

Recognizing the importance of prompt seizure management, healthcare organizations and professionals are actively working to improve awareness, develop effective assessment tools, and promote a patient-centered approach to care. By increasing understanding and implementing appropriate strategies, healthcare providers may better support individuals living with seizures, minimize the impact of clinical issues, and enhance overall patient outcomes. For example, seizure activity may be accurately classified using electroencephalogram (EEG) signals and predictive models as described herein. In particular, the methods and systems described herein may improve upon conventional methods and systems for seizure classification by discriminating between electrographic seizures, highly pathological EEG with high likelihood of epileptiform activity, and normal electrographic activity in classifying a subject's brain activity over a period of time.

In an aspect, the present disclosure provides a method for detecting seizure in a subject including: obtaining data comprising EEG signals recorded from a subject over a plurality of channels; pre-processing the data by: dividing the data into a plurality of temporal segments, and extracting a plurality of features from each of the temporal segments; generating a preliminary seizure classification for each of the temporal segments based on the plurality of extracted features; and determining an overall seizure classification for the subject based on the preliminary seizure classification for each of the temporal segments.

In an aspect, the present disclosure provides a system for classifying seizure including: a data module configured to receive data having a plurality of EEG signals recorded during a time window and over a plurality of channels from a subject; and a seizure detection module having a memory storing a set of instructions and one or more processors that are configured to, responsive to the set of instructions: pre-process the data received by the data module by: dividing the EEG signal into a plurality of temporal segments, and extracting a plurality of features from each of the plurality of temporal segments; generate a preliminary seizure classification for each of the temporal segments based on the plurality of extracted features; and determine an overall seizure classification for the subject based on the preliminary seizure classification for each of the temporal segments.

### Classification System

I. Signal acquisition and pre-processing
II. Signal analysis
III. Neurological condition detection and output
IV. Seizure classification
V. Post neurological condition detection
VI. Computer systems

### I. Signal Acquisition and Pre-Processing

For ease of explanation, the figures and corresponding description below are described below with reference to analysis of signals representing brain activity (e.g., electroencephalography (EEG) signals) and/or heart activity (e.g., electrocardiogramaignals) of a living subject. However, one of skill in the art will recognize that signals representing other bodily functions (e.g., an electromyography (EMG) signal, or an electronystagmography (ENG) signal, a pulse oximetry signal, a capnography signal, and/or a photoplethysmography signal) may be substituted, or used in addition to (e.g., in conjunction with), one or more signals representing brain activity and/or heart activity. In some variations, the signals are EEG signals analyzed to detect and classify seizure or epileptiform activity in a patient.

A system for measuring bioelectrical signals may generally comprise one or more electrodes electrically coupled via corresponding conductive wires to a controller and/or output device. In other variations, the electrodes may be coupled to the controller and/or output device wirelessly. The electrodes may be contained within an electrode carrier system that is secured around the head of the patient. The electrode carrier system may be configured as a headband or incorporated into any number of other platforms or positioning mechanisms for maintaining the electrodes against the patient body. Individual electrode assemblies may be spaced apart from one another so that, when the headband is positioned upon the patient's head, the electrode assemblies may be aligned optimally for receiving EEG signals. In some variations, the electrode carrier system may be used to detect and classify seizure and/or epileptiform activity in a patient.

In some variations, EEG signals from 10 electrodes may be combined. The locations of the electrodes may be, for example, Fp1, Fp2, F7, F8, T3, T4, T5, T6, O1, and O2. These electrodes may form 8 channels (Fp1-F7, F7-T3, T3-T5, T5-01, Fp2-F8, F8-T4, T4-T6, and T6-O2, or any combination thereof).

The number of channels from which EEG signals are obtained and recorded may range from 1 to 45, including all values and sub-ranges therein. For example, the plurality of channels may include 1 channel, 2 channels, 3 channels, 4 channels, 5 channels, 6 channels, 7 channels, 8 channels, 9 channels, 10 channels, 11 channels, 12 channels, 13 channels, 14 channels, 15 channels, 16 channels, 17 channels, 18 channels, 19 channels, or 20 channels, 22 channels, 23 channels, 24 channels, 25 channels, 26 channels, 27 channels, 28 channels, 29 channels, 30 channels, 31 channels, 32 channels, 33 channels, 34 channels, 35 channels, 36 channels, 37 channels, 38 channels, 39 channels, 40 channels, 41 channels, 42 channels, 43 channels, 44 channels, or 45 channels. In one variation, an 8 channel EEG may be used to classify seizure and/or epileptiform activity.

In certain embodiments, as further described herein, each channel may be assigned to an independent machine learning model, and the extracted features applied to the machine learning model corresponding to the channel.

The electrodes may be part of an electrode assembly and electrode carrier system, as mentioned above. The electrode carrier system may generally comprise an electrode body which is at least partially electrically conductive, one or more members (e.g., one or more tubular members) extending from the electrode body, each of the one or more members defining a lumen therethrough and a distal opening, a reservoir having a compressible structure and containing a conductive fluid or gel which is in fluid communication with the one or more members, and a backing supporting the electrode body and reservoir.

In some variations, the electrode carrier system may generally comprise an electrode body having one or more tubular members extending therefrom, each of the tubular members defining a lumen therethrough and a distal opening, a reservoir having a compressible structure which defines an internal volume and which is in fluid communication with the one or more tubular members, and a controller and/or output device which is in electrical communication with the electrode body, wherein the controller and/or output device is configured to receive electrical signals from the electrode assembly and record and/or output a corresponding response.

The electrode carrier system may generally comprise a backing secured around the head of a patient. The backing may be configured as a headband although the carrier system may be incorporated into any number of other platforms or positioning mechanisms for maintaining the electrodes against the patient body. The individual electrodes are spaced apart from one another so that when the headband is positioned upon the patient's head, the electrodes are aligned optimally upon the head for receiving EEG signals. The carrier system may have each of the electrodes electrically coupled via corresponding conductive wires extending from the backing and coupled, e.g., to a controller and/or output device. Although in other variations, the electrodes may be coupled to the controller and/or output device wirelessly.

The controller and/or output device may generally comprise any number of devices for receiving the electrical signals such as electrophysiological monitoring devices and may also be used in combination with any number of brain imaging devices, e.g., fMRI, PET, NIRS, etc. In one particular variation, the electrode embodiments described herein may be used in combination with devices such as those which are configured to receive electrical signals from the electrodes and process them.

In one variation, the electrode carrier system may comprise each of the electrodes enclosed within a reservoir which is pre-filled with a conductive gel or fluid. Each electrode may be configured into a flattened or atraumatic configuration which is contained within a respective reservoir and each reservoir may be formed of any number of flexible materials, e.g., silicone, polyurethane, rubber, etc., which may readily collapse. The electrodes may be coupled via conductive wires passing through a lumen defined through the backing separated from the electrodes by a substrate. Each reservoir may also respectively define one or more openings through which the conductive gel or fluid may be expelled.

Once the platform has been situated over the patients' head, the user may press upon each of the reservoirs such that the conductive fluid or gel flows through the openings and onto the skin of the patient. The conductive fluid or gel expelled through the openings may maintain fluid communication between the skin surface and the respective electrodes such that the detected electrical signals may be transmitted from the skin and to the electrodes. Moreover, because of the flexibility of the reservoirs, once the conductive fluid or gel has been expelled into contact with the skin surface, the backing may lie flat against the skin surface so that the patient may comfortably lay their head upon a surface while still maintaining electrical contact with the electrodes.

Another electrode variation may be comprised of one or more loops of conductive wire or ribbon which are able to readily bend or flex against the skin surface. The electrode carrier system may include a pressure release reservoir for containing the conductive fluid or gel, as described above, around each of the electrodes so that the conductive fluid or gel may be expelled around and within the one or more loops to ensure a conductive path.

In further variations of the electrode assembly, one or more tubular members may extend from the backing transversely. The tubular members may be each arranged in a circular pattern for each electrode and they may also define a lumen therethrough with an opening defined at each distal end. Each of the tubular members may be fabricated from a conductive metal which may retain its tubular shape when in use or which may be sufficiently thin and flexible to bend or yield when placed against the patient's skin surface. Alternatively, the tubular members may be fabricated from a flexible material which is coated or layered with a conductive material such that the members retain their flexibility. In either case, the conductive fluid or gel may be either contained within the tubular members or they may be retained within a pressure release reservoir, as described above, surrounding or in proximity to each electrode. Because of the tubular shape of the electrodes, they may readily pass through the patient's hair, if present, and into contact against the skin surface while maintaining electrical contact.

Yet another variation of an electrode embodiment may also utilize a pressure release reservoir filled with the conductive fluid or gel. The reservoir may be formed of a flexible material, e.g., silicone, polyurethane, rubber, etc., extending from the backing to form a curved or arcuate structure with one or more openings defined over the reservoir. These openings may remain in a closed state until a force is applied to the reservoir and/or backing which may urge the fluid or gel contained within to escape through the openings and into contact with the outer surface of the reservoir and underlying skin surface. The outer surface of the reservoir may have a layer of conductive material in electrical contact with the conductive wires so that once the fluid or gel has been expelled from within the reservoir and out onto the conductive material upon the reservoir outer surface and skin surface, electrical contact may be achieved.

The electrode carrier system in some instances may include an electrode body that may define one or more tubular members extending from the body such that the members project transversely away from the backing. The electrode body may be comprised of a conductive material such as a metal which may be rigid. However, in other variations, the body may be fabricated from a conductive material which is also flexible, e.g., conductive silicone, and/or from a flexible material, e.g., silicone, polyurethane, rubber, etc., which may be coated or layered with a conductive material such that the underlying tubular members retain their flexibility. In either case, the body may be secured to the backing such that the one or more openings are defined along the body and extending through the members are in fluid communication with a reservoir having a compressible housing. The reservoir may also be secured to the backing and contain a volume of conductive fluid or gel local to the electrode body.

The tubular members may be arranged in various patterns, e.g., a circular pattern, a uniform pattern, or in an arbitrary pattern. When the backing has been secured to the patient, the reservoir may be pressed or urged such that the fluid or gel contained within is expelled through each of the tubular members and into contact against the underlying skin surface through corresponding distal openings. The elongate nature of the members may enable them to pass readily through the patient's hair, if present, and into direct contact against the skin surface.

In another variation, an electrode carrier system having a tubular body may define one or more openings over its surface. The tubular body may have one or more tubular members which extend in a spiral or helical pattern away from the backing. The tubular members may define a lumen therethrough which extends from the tubular body and to a distal opening at its tip. The backing may further define a reservoir which contains a volume of conductive fluid or gel such that the body is in fluid communication with the reservoir. In some variations, the distal tips of the members may present a roughened surface for contacting the skin. The optionally roughened tips may be rotated or otherwise translated or moved across over the skin surface by the user to at least partially exfoliate the skin surface to facilitate electrical contact.

For example, a distal skin-contacting surface of the electrode assembly may be modified to prepare the skin surface to enhance electrical conductance (i.e., lower electrical resistance) between an electrically conductive portion of the electrode assembly and the skin when that electrically conductive portion is in physical contact with the skin. For example, the tissue-contacting surface(s) of the electrode assembly may be modified to have an abrasive surface, e.g., by coating with abrasive particulate; may be formed or molded to have protruding rigid features, e.g., bumps, ridges, or the like; and/or may be coated with a material that lowers the electrode connection impedance. Such sweeping and/or chemical coating of the tissue-contacting surface(s) of the electrode assembly over the target tissue location could scrub, dissolve and/or otherwise disrupt dead tissue and break-up scalp oil. In specific examples, at least a portions of a distal tissue-contacting surface of the electrode assembly, for example the distal surface(s) of at least some of the tubular members, comprise such surface features, surface coatings, surface treatments, or combination thereof to improve the quality of the electrode connection.

In another embodiment, an electrode assembly comprises an electrode body and one or more tubular members extending from the electrode body, typically from a bottom surface of the electrode body. Each tubular member has a distal tip, and at least some of the tubular members have a lumen with a distal opening in the distal tip. A reservoir containing a conductive fluid or gel is optionally disposed in the electrode body, and the electrode body is configured for dispensing the conductive fluid or gel from the reservoir through the lumen(s) and out of the distal opening(s) of the tubular member(s). Alternatively, in some embodiments, the conductive fluid or gel may be dispended onto or through the lumens of the tubular member using a syringe or other separate delivery device.

In specific embodiments, the electrode assembly will typically comprise at least two tubular members, and may comprise three tubular members, four tubular members, or even more. The tubular members will usually depend vertically downwardly from a bottom surface of the electrode body and will be specifically configured so that they may penetrate a patient's hair so that a distal tip of the tubular members will be able to engage and provide reliable electrical contact with a patient's scalp. The tissue engagement areas of the tubular members on bottom surface of the electrode body will usually be 50% or less of the area of the bottom surface, frequently being 30% or less of the area of the electrode body, and usually being at least 5% of the area of the bottom surface. Thus, the tissue engagement areas of the tubular members on bottom surface of the electrode body will usually be in a range from 5% to 50% of the area of the bottom surface, typically being in a range from 5% to 30% of the area of the bottom surface.

In most instances, the tubular members will extend from a generally planar bottom of the electrode body at a perpendicular angle. In other instances, however, the tubular members may extend at an angle anywhere in the range from 30° to 150° relative to the plane, typically being from 60° to 120° relative to the plane. In other instances, however, the tubular members may have other configurations, for example being configured in a helical shape so that they may penetrate hair to a patient's scalp by rotating the electrode assembly around a vertical axis.

In other embodiments, the distal tips of at least some of the tubular members will have a skin preparation, e.g., tissue-roughening, surface. For example, the tissue-roughening surface may comprise an abrasive material, such as a grit or other abrasive particles, formed over at least a portion of the distal tip of the tubular member. In other instances, the surface-roughening may comprise surface features, such as ridges, bumps, grooves, and the like, formed over at least a portion of the distal tip which contacts the patient's skin.

The electrode body, and in particular the tubular members connected to the electrode body, may be formed at least partly from electrically conductive materials, such as metals, electrically conductive coatings, embedded wires, or electrically conductive polymers. In such instances, the electrode body and/or the tubular members will provide at least a portion of the electrical path needed to conduct biological currents from the tip of the tubular member(s) to an electrical terminal or other conductive connector on the electrode body as described below. In other instances, however, the electrode body and/or the tubular members may be formed primarily or even entirely from an electrically non-conductive material. In such instances, the electrically conductive fluid or gel will provide most or all of the electrically conductive path needed to deliver the biological current from the distal tip of the tubular member to the electrical terminal after such conductive fluid or gel has been distributed throughout the electrode body and tubular member.

The members may comprise a variety of geometries. In some instances, the tubular members may be generally cylindrical having a lumen extending therethrough. In other instances, however, the tubular members may be formed as “prongs” having a relatively broad tissue-contacting region along a curved “axis” at their distal tips. In many instances, the tissue-contacting regions of the prongs will be generally crescent-shaped so that they will follow a generally circular path as they are rotated against the patient's tissue.

The prongs and other members (e.g., tubular members) may have a port in their tissue-contacting surfaces for delivering the electrically conductive fluid or gel to the patient's skin. In some instances, ports may be formed in a generally flat bottom surface of the tubular members or prongs. In other instances, the ports may be connected to a channel or other distribution feature on the tissue-contacting surface of the prong or other tubular member. In still further specific embodiments, the ports for delivering the electrically conductive fluid or gel may be located in a recessed surface of the prong which may adjacent to a tissue-contacting lower surface of the prong or other tubular member.

While the electrode assemblies will usually comprise one or more members (e.g., prongs, tubular members) as just discussed, in some alternative embodiments, the electrode body may have a generally flat bottom free from tubular and other protruding members. The flat bottom may be configured to engage the skin and have openings to release a conductive fluid or gel in any of the ways described elsewhere herein for delivering the conductive fluid or gel. The tissue-contacting surface(s) of such flat bottoms may be modified in any of the ways discussed herein, e.g., roughened or textured, to have electrical conductivity with the target tissue surface(s).

In use, the plurality of electrodes may be placed on patient's scalp by placing a headband or other headgear around the patient's scalp. The headband carries a plurality of electrode assemblies, for example as described above, and distal tip(s) of one or more tubular members extending from at least some of the electrode assemblies may be engaged against scalp tissue. An electrically conductive fluid or gel may then be extruded from a reservoir disposed in at least some of the electrode assemblies so that the fluid or gel passes through the tubular members to form an electrically conductive path to the patient's scalp tissue. The plurality of electrode assemblies may then be connected to a controller and/or output device configured to receive low power biological current from the electrode assemblies. In some variations, at least some of the plurality of electrode assemblies have roughened surfaces that may be rotated in order to abrade scalp tissue adjacent the distal tip(s) of said one or more tubular members in order to lower contact resistance between the electrode assembly and the scalp tissue.

In some embodiments, signals corresponding to brain electrical activity are obtained from a human brain and correspond to electrical signals obtained from a single neuron or from a plurality of neurons. In some embodiments, sensors include one or more sensors affixed (e.g., taped, attached, glued) externally to a human scalp (e.g., extra-cranial sensor). For example, an extra-cranial sensor may include an electrode (e.g., electroencephalography (EEG) electrode) or a plurality of electrodes (e.g., electroencephalography (EEG) electrodes) affixed externally to the scalp (e.g., glued to the skin and using conductive gel to form electrical contact), or more generally positioned at respective positions external to the scalp Alternatively, dry electrodes may be used in some implementations (e.g., conductive sensors that are mechanically placed against a living subject's body rather than planted within the living subject's body or contacted through a conductive gel). An example of a dry electrode is a headband with one or more metallic sensors (e.g., electrodes) that is worn by the living subject during use. The signals obtained from an extra-cranial sensor may sometimes be called EEG signals or time-domain EEG signals. In some cases, a sensor may be an accelerometer or an inertial measurement unit (IMU) that may measure the mechanical movement of the subject and/or the device (e.g., produce one or more electrical signals corresponding to mechanical movement of the subject and/or device). The system may be configured to utilize one or more sensors to aid in determining a potential neurological condition as described elsewhere herein.

### Neurological Condition Detection and Monitoring System-Data Module

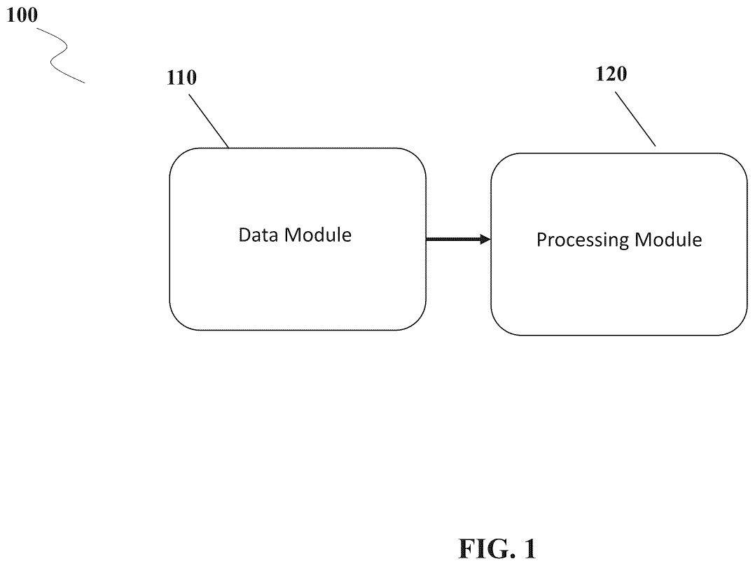

In an aspect, the present disclosure provides a seizure classification system 100. As shown in FIG. 1, the system 100 may include a data module 110 configured to obtain data. The data obtained by the data module 110 may include a plurality of electroencephalography (EEG) signals collected from a subject. The data may also include non-EEG data. The non-EEG data may include blood pressure, heart rate, and/or motion data of the subject. The non-EEG data may be as described elsewhere herein.

In another aspect, the present disclosure provides a seizure classification method. The seizure classification method may include obtaining data. The data may include a plurality of electroencephalography (EEG) signals collected from a subject. The method may include processing the data to (1) detect seizure or epileptiform activity that the subject has experienced or is experiencing, and (2) generate indications or assessments for the epileptiform activity.

The data module 110 may include a plurality of electrodes that are configured to be placed on different regions of the subject's head. The different regions may include frontal lobes, temporal lobes, and/or occipital lobes. The data module may also include a plurality of channels that multiplexes the EEG signals from the plurality of electrodes in each region and between the different regions. The electrodes may be used on the frontal lobes of the patient. The location of electrodes may be, for example, Fp1, Fp2, F7, F8, T3, T4, T5, T6, 01, 02 and channels (Fp1-F7, F7-T3, T3-T5, T5-O1, Fp2-F8, F8-T4, T4-T6, and T6-O2, or any combination thereof. In some cases, having electrodes at multiple locations may provide more coverage, more channels, more tolerance for noise or artifact from a specific channel, and capable of monitoring effects of different agents that affect different parts of the brain. In some cases, having electrodes at multiple locations may allow for more accurate or precise determination of epileptiform activity. The number of channels provided by the electrodes may range, for example, between 1 and 45, as previously described herein. It may be beneficial to employ 8 channels for seizure classification.

In some embodiments, the data module 110 may have one or more analog front ends configured to receive sensor EEG signals from sensors. The EEG signals may be preprocessed as described elsewhere herein. In some embodiments, a separate (e.g., independent) analog front end may be provided for interfacing with each of a set of sensors. In some embodiments, one or more analog front ends may be provided for interfacing with a set of EEG sensors.

### Neurological Condition Detection System-Processing Module

The system may include a processing module 120 in communication with the data module 110. The processing module 120 may be configured to process the data (e.g., EEG signals) to detect and classify brain activity (e.g., seizures or epileptiform activity) that the subject has experienced or is experiencing. The processing module 120 may generate indications or assessments for seizures. The indications or assessments may be presented to a user via a notification output module 290.

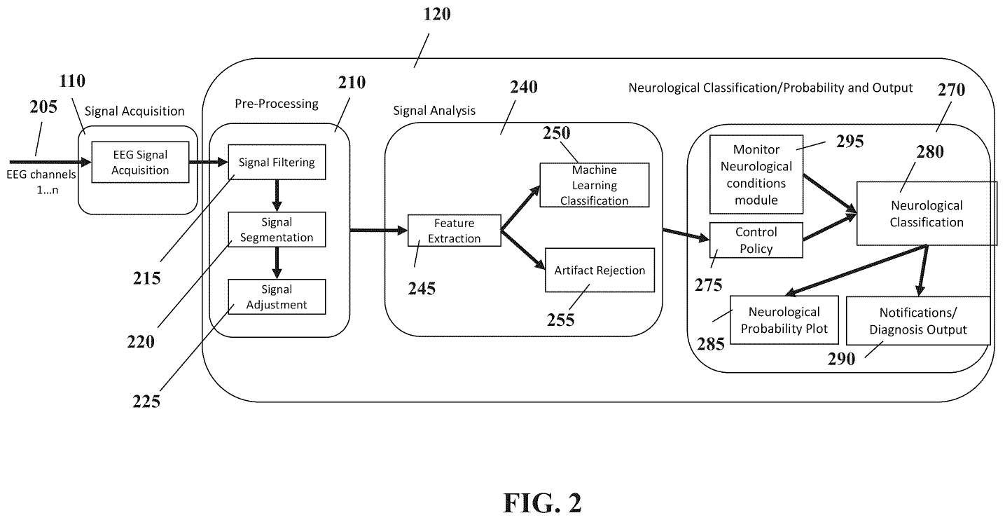

As shown in FIG. 2, the processing module 120 may be configured to process the data from the data module 110 to simultaneously detect and monitor the one or more neurological conditions in real-time. The brain activity may relate to, for example, a seizure state (e.g. seizure-like or epileptiform activity) or a non-seizure state (e.g., normal activity).

### Preprocessing of EEG Signals

In some embodiments, the method may include preprocessing the plurality of signals by segmenting the plurality of signals for each channel into a plurality of temporal data segments. As shown in FIG. 2, the processing module 120 may include a pre-processing module 210. In some embodiments, the method may include preprocessing the plurality of EEG signals by segmenting the plurality of EEG signals for each channel into a plurality of temporal data segments. FIG. 2 shows an illustration of the processing module 120. The processing module intakes EEG signals from a plurality of channels from the data module 110. The processing module may preprocess the EEG signals from a plurality of channels with a preprocessing module 210 configured to preprocess EEG signals. As shown in FIG. 2, the preprocessing module may include a signal filtering module 215, signal segmenting module 220, and signal adjustment module 225.

In some embodiments, the filtering module 215 may be configured to may filter EEG signals from the incoming set of channels from the EEG device module as described elsewhere herein. In some cases, preprocessing may be, for example, segmenting the EEG signals, filtering the EEG signals based on frequency, adjusting the EEG signals, or as described elsewhere herein, etc.

In FIG. 2, the signal segmentation module 220 may be configured to segment EEG signals. Each temporal data segment of the EEG signal (which may be referred to herein as a “temporal segment”) may be associated with a given time epoch. Each time epoch may be defined by a start time and a duration. Multiple EEG signals from different EEG electrodes may be segmented to have temporal segments that correspond the same time epoch, so that they may be, by way of example, analyzed subsequently to detect temporal correlations in their respective waveforms. For each corresponding time epoch, a cluster of neurological condition-positive classifications based on an analysis of features extracted from the corresponding temporal segments as described herein below may be indicative of a category of seizure activity (e.g., seizure-like activity, epileptiform activity, or normal activity).

In some embodiments, the plurality of EEG signals may be segmented to between 1 to 100000 data segments. In some cases, the number of EEG data segments may depend on the duration of the EEG recordings. In some cases, the number of EEG data segments may be fixed regardless of the duration of the EEG recordings.

In some embodiments, each temporal data segment may have a duration of between about 1 second to 1 hour. In some cases, each temporal data segment may have a duration of between about 1 second to 30 seconds. In some cases, each temporal data segment may have a duration of between about 1 second to 10 seconds. In some cases, the duration of each temporal data segment may be fixed for the entire EEG recording. In some cases, the duration of each temporal data segment may be variable or adaptive during an EEG recording.

In some embodiments, the preprocessing of the plurality of EEG signals may comprise applying one or more filtering steps to the plurality of EEG signals over the plurality of channels. The preprocessing of the plurality of EEG signals may comprise using at least 1 filter, 2 filters, 3 filters, 4 filters, 5 filters, 6 filters, 7 filters, 8 filters, 9 filters, 10 filters, 15 filters or more. The preprocessing of the plurality of EEG signals may comprise using at most about 15 filters, 10 filters, 9 filters, 8 filters, 7 filters, 6 filters, 5 filters, 4 filters, 3 filters, 2 filters or less. The preprocessing of the plurality of EEG signals may comprise using anywhere between 1 to 15 filters, 1 to 10 filters, 1 to 5 filters, or 1 to 3 filters.

In some embodiments, the one or more filtering steps may be applied before, during, and/or after the segmentation of the plurality of EEG signals. One or more of the filtering steps may include, for example, a digital filter, an analogue filter, or a combination thereof. One or more of the filtering steps may include, for example, a bandpass filter, low-pass filter, a high-pass filter, a band-stop filter, an all-pass filter, a Kalman filter, an adaptive filter, or a notch filter, etc. In some cases, the low frequency cutoff of the filters may be between 0.1 Hz and 5 Hz. In some cases, the high frequency cutoff of the filters may be between 5 Hz and 200 Hz. In some cases, the notch filter frequency may match the local power line frequency. In some cases, the notch filter frequency may be 50 Hz or 60 Hz to match the local power line frequency.

For seizure classification, an 8-Channel EEG may be generated from raw waveforms recorded at a sampling rate of 250 Hz. The signals may be band-pass filtered between 1 Hz and 35 Hz using a 5th order Butterworth filter. In some variations, subsequent high-pass filtering may be used for better removal of DC components for some of the feature calculations, as further described below. The filtered EEG data may then be segmented into window sizes (durations) ranging from about 1 second to about 10 minutes, including all values and sub-ranges therein. In some instances, the window size may be greater than 10 minutes. The window size may be about 1 second, about 2 seconds, about 3 seconds, about 4 seconds, about 5 seconds, about 10 seconds, about 15 seconds, about 20 seconds, about 25 seconds, about 30 seconds, about 35 seconds about 40 seconds, about 45 seconds, about 50 seconds, about 55 seconds, about 60 seconds, about 2 minutes, about 3 minutes, about 4 minutes, about 5 minutes, about 6 minutes, about 7 minutes, about 8 minutes, about 9 minutes, or about 10 minutes with an overlap of 0% to 95% (between consecutive windows). For example, in some variations, the filtered EEG data may be segmented into 15 second windows with 33% (5 second) overlap between consecutive windows. In other variations, the filtered EEG may be segmented into 10 second windows with no overlap, or 4 second windows with 75% (3 seconds) overlap, or 60 second windows with 50% (30 seconds) overlap.

### EEG Signal Adjustment

FIG. 2 shows a signal adjustment module 225 configured to adjust an EEG signal. In some embodiments, the method may adjust any EEG signal. Adjusting an EEG signal may include, for example, increasing and/or decreasing the amplitude of the EEG signal, adding or decreasing the noise level of the EEG signal, increasing and/or decreasing a time epoch of the EEG signal, increasing and/or decreasing the intensity of the EEG signal, increasing and/or decreasing the signal frequency of the EEG signal, increasing and/or decreasing the voltage of the EEG signal, changing the morphology of the EEG signal (e.g. the shape of the EEG signal), increasing and/or decreasing the periodicity of the EEG signal, increasing or decreasing the synchrony of the EEG wave, spectral subtraction, standardizing etc.

In some cases, the EEG signal may be reduced. In some cases, the EEG signal may be downsampled to a lower sampling frequency. For example, EEG data recorded at a sampling frequency of 500 Hz may be down sampled by a factor of 2 to 250 Hz.

In some cases, the EEG signal may be subjected to bit-width reduction. In some cases, the level of resolution at which the EEG signals are recorded may not be required by the method to accurately generate seizure classifications. In some cases, the bit-width reduction may reduce the EEG signal to a lower number of bits per sample through standard quantization of the EEG signal, for example, from 32 bits per sample to 12 bits per sample. In some cases, bit-width reduction may be advantageous if the method is to be implemented in a portable system, as it may be useful for reducing power consumption due to decreased processing load.

In some cases, spectral subtraction may be used to reduce the amount of additive noise in the EEG signal. In some cases, the noise may be caused by external surroundings. In some cases, the noise may be caused by the measurement equipment. In some cases, the noise may be caused by the user. In some cases, an average frequency spectrum of non-stroke EEG signal may be computed over a period of time to provide a base level estimate of the noise frequency spectrum. In some cases, as the EEG signals are recorded, the EEG signals may be converted to the frequency domain. In some cases, the average noise spectrum may then be subtracted from the EEG frequency spectrum. In some cases, the resulting spectrum and phase information from the original noisy signal may be combined. In some cases, the resulting spectrum may be transformed back into time domain to produce a de-noised signal.

In some embodiments, the EEG signal may be standardized by eliminating the effect of the montage that was used in gathering the EEG signals. In some cases, independent component analysis (ICA) or principal component analysis (PCA) methods may be used to provide the montage elimination. In some cases, the ICA or PCA method may separate the EEG signal into a set of sources independent of the montage used to record them. In some cases, using standardized EEG data may remove errors introduced by the varying practices of clinicians.

In some cases, a non-negative matrix factorization (NMF) method may be applied to each channel as a form of artifact removal. In some cases, the spectrum of the signal may be decomposed into the extracted bases to obtain weights. In some cases, the spectrum may be reconstructed using the bases of artifacts and the corresponding weights removed from the initial EEG signal. In some cases, independent component analysis (ICA) or principal component analysis (PCA) methods may be used for artifact reduction or removal.

### II. Signal Analysis

The processing module 120 may be configured to process the data to detect and analyze a plurality of features that are likely to be associated with seizure-like and epileptiform activity. The processing module 120 may be configured to use the plurality of features as inputs to train a predictive model (e.g., a machine learning algorithm) for classifying different seizure types or severities.

### Feature Extraction

In FIG. 2, the processing module may comprise a signal analysis module 240. The signal analysis module 240 may comprise a feature extraction module 245 and a machine learning classification module 250. The feature extraction module 245 may be configured to take preprocessed measured data (e.g., EEG signals or temporal segments of the EEG signals of a given time epoch) from the preprocessing module 210 to build derived values (e.g., features).

The plurality of features extracted by feature extraction module 245 may include a plurality of time-domain features and frequency-domain features. The plurality of features may include a plurality of time-domain features and frequency-domain features of the data provided from the data module 110 and/or pre-processing module 210. The plurality of features may include brain asymmetry, amplitude variations, spatial and temporal correlations, coherences, or co-variations of two or more features. The plurality of features may be ranked and/or classified.

In some embodiments, feature extraction may start from an initial set of measured data (e.g., EEG signals or temporal segments of EEG signals of a given time epoch, etc.) and may build derived values (e.g., features) intended to be informative and non-redundant. In some cases, the feature extraction module may include extracting a plurality of features from each temporal data segment for each channel individually. In some cases, the feature extraction module may include extracting a plurality of features from each temporal data segment for all channels together. In some cases, the feature extraction module may include extracting a plurality of features from each temporal data segment of one or more groupings with each grouping consisting of one or more channels.

As shown in FIG. 2, the extracted features may be relayed to a machine learning classification module 250 that may be configured to analyze and classify the extracted features as described elsewhere herein. In some cases, feature extraction may facilitate the subsequent learning and generalization steps of a machine learning algorithm. In some cases, feature extraction may lead to better human interpretations. In some cases, feature extraction may be related to dimensionality reduction.

In some cases, when the input data (e.g., EEG signals) to the machine learning algorithm is too large to be processed and suspected to be redundant (e.g., the same measurement in both Hz and seconds, or the repetitiveness of a characteristic), the data may be transformed into a reduced set of features.

In some cases, determining a subset of the initial features may be called feature selection. In some cases, the selected features may be expected to contain the relevant information from the input data (e.g., EEG signals or temporal segments of the EEG signals). In some cases, the selected features may be expected to contain the relevant information from the input data so that the desired task may be performed by using this reduced representation instead of the complete initial data.

In some embodiments, feature extraction may involve reducing the number of resources required to describe a large set of data (e.g., EEG signals or temporal segments of the EEG signals). In some cases, analysis with a large number of variables may require a large amount of memory and computation power. In some cases, feature extraction may construct combinations of the variables to accurately describe the data with sufficient accuracy. In some cases, feature extraction may construct combinations of the variables to accurately describe the data with sufficient accuracy while preventing overfitting.

In some embodiments, results may be improved using constructed sets of application-dependent features. In some cases, the constructed sets may be built by an expert. In some cases, general dimensionality reduction techniques may be used. In some cases, general dimensionality reduction techniques may be, for example, independent component analysis, isomap, kernel PCA, latent semantic analysis, partial least squares, principal component analysis, multifactor dimensionality reduction, nonlinear dimensionality reduction, multilinear principal component analysis, multilinear subspace learning, semidefinite embedding, autoencoder, etc.

In some cases, a set of numeric features may be described by a feature vector. In some cases, a feature vector may be an n-dimensional vector of numerical features that represent some object, by way of example an EEG signal or a temporal segment of the EEG signal.

In some embodiments, data analysis software packages may provide for feature extraction. In some cases, data analysis software packages may provide for dimension reduction. In some cases, data analysis software packages may include programming environments such as MATLAB, SciLab, NumPy, or the R language, etc. In some cases, a programming language script may be used to extract features from EEG signals. In some cases, the programming language script may be, for example, MATLAB, Python, Java, JavaScript, Ruby, C, C++, or Perl, etc.

In some cases, the plurality of features may be intrinsic in the plurality of EEG signals or temporal segments thereof. Intrinsic may be a feature of an EEG signal that may be measured, for example, the amplitude of the EEG signal, the duration of the EEG signal, the variation of the EEG signal, the power of the EEG signal, the local maxima/minima of the EEG signal, the pattern of the EEG signal, the regularity of the EEG signal, the spectral power distribution of the EEG signal, or the frequency of the EEG signal, etc. In some cases, the plurality of features may be a measurement of the power of a signal within a particular frequency band. The frequency band may be, for example, from about 0 Hz to 100 Hz. In some cases, the power of a signal may be normalized to the total power. In some cases, the power of a signal may be a ratio of power between one or more frequency bands. In some cases, a feature may be a function performed on a signal to obtain a value. For example, a function may measure the root mean square (RMS) of a signal (e.g., EEG signal) to obtain the RMS value of the signal. In some cases, a feature may compare one signal (e.g., EEG signal) to one or more signals. In some cases, a feature may compare one or more signals (e.g., EEG signals) to one or more signals. In some cases, a feature may measure an attribute of a signal (e.g., EEG signal). In some cases, a feature may compare one or more attributes of a signal (e.g., EEG signal) with one or more attributes of a signal. An attribute may be, for example, an intrinsic property of the EEG signal. In some case, the feature of an EEG signal may be continuous and/or discrete in time.

In some cases, the plurality of features may include at least twenty different time and/or frequency domain features. In some cases, the plurality of features may include at most one thousand time and/or frequency features. In some cases, the plurality of features may include between about 10 features to 200 features. In some cases, the plurality of features may include between about 10 features to 100 features. In some cases, the plurality of features may include between about 10 features to 50 features.

In some cases, the plurality of features may include a plurality of discrete values associated with the time domain, frequency domain, time-frequency domain, information theory, and nonlinear-dynamics system theory features. In some cases, the plurality of features may include a plurality of discrete values associated with the time and/or frequency domain features. The plurality of features may include a plurality of continuous values associated with the time and/or frequency domain features or the morphology of the signal. In some cases, the plurality of features may also be brain asymmetry, amplitude variations, spatial and temporal correlations, coherences or co-variations of two or more features. In some cases, the plurality of features may also be frequency spectrum and characteristics of the EEG signal(s). In some cases, characteristics of the signal may include, jitter, skew, spread spectrum, time measurements, frequency measurements, etc. In some cases, analyzing the plurality of features may include ranking and/or classifying the plurality of features.

In some cases, the plurality of signals may be converted into a digital signal. In some cases, the plurality of signals may be converted into a digital signal and then an analog signal.

In some cases, the features may be sampled from a portion of the EEG signal. Features may be sampled from a portion of the EEG signal to reduce processing time and power required.

In some embodiments, a feature may be pertaining to a certain weight value. The weight value may give one feature a higher score for detecting a neurological condition or a particular neurological condition. The higher score may indicate that the feature may be more relevant in predicting one or more neurological conditions. The score may indicate that the feature may be more relevant in predicting a certain neurological condition. The score may indicate that the feature may be more relevant in predicting the severity of a particular neurological condition. The method may adjust the weight value of any feature at any given time. The method may adjust by increasing and/or decreasing the weight value of any feature at any given time.

In variations where seizure is to be detected and classified, for each temporal segment, e.g., each 4 second, each 10 second, each 15 second, each 30 second, or each 60 second segment, features may be computed using time-frequency analysis of data recorded on individual channels to produce single-channel features. Additionally, a set of features may be computed to analyze signal interactions between pairs of channels, called multi-channel features. Exemplary single channel features may include power in different frequency bands (for example, Alpha, Beta, Delta, Theta, and Gamma), spectral properties, power ratios, amplitude characteristics and morphology features, entropy, variability and wavelet decomposition. Exemplary multi-channel features that may be computed to quantify inter-channel interactions may include correlation within and across hemispheres for different frequency bands (for example, Alpha, Beta, Delta, Theta, and Gamma), as well as spectral, amplitude and phase related correlations and synchrony measures.

A temporal segment of EEG data may be marked as an artifact if a predefined set of features cross certain threshold values. Further, if the most recently reported impedance on an electrode is higher than a preset threshold, the segment may also be marked as an artifact. If an EEG window is marked as an artifact, it is generally not used for subsequent analysis and predictions.

### Classification Using Predictive Models

In some embodiments, the method may include applying a predictive model (e.g., a machine learning algorithm) to the plurality of features to perform a classification to one or more neurological conditions for each temporal data segment for each channel individually. In some cases, the predictive classification module may include performing classification to one or more neurological conditions for each temporal data segment for all channels together. In some cases, the predictive classification module may include performing classification to one or more neurological conditions for each temporal data segment of one or more groupings with each grouping consisting of one or more channels. FIG. 2 shows the predictive classification module 250 that may take the features collected/extracted from the preprocessing step and classify the features. In some cases, the features may be extracted without a preprocessing step.

In some cases, predictive models may need to extract and draw relationships between features as conventional statistical techniques may not be sufficient. In some cases, predictive models may be used in conjunction with conventional statistical techniques. In some cases, conventional statistical techniques may provide the predictive model with preprocessed features.

In some embodiments, the plurality of features may be used by one or more predictive models to provide a classification for a temporal segment with respect to a neurological condition.

In some embodiments, a cluster of neurological indicative-positive classifications may comprise of between about 1 to 50 neurological indicative positive classifications. In some cases, a cluster of positive classifications may comprise of between 1 to 10 neurological indicative-positive calculations.

In some embodiments, the method may further comprise comparing the classifications sequentially across a plurality of time epochs on each channel. In some cases, before/after/during comparing the classifications sequentially across a plurality of time epochs on each channel, the classifications sequentially across a plurality of time epochs on each channel may be discarded. In some cases, a subset of the classifications may be discarded. In some cases, a subset of fewer than about 1 to 20 classifications may be discarded. In some cases, a subset of fewer than 3 classifications may be discarded. In some cases, a subset of fewer than 7 classifications may be discarded. In some cases, a subset of fewer than 10 classifications may be discarded. In some cases, a subset of fewer than 15 classifications may be discarded. In some cases, a subset of fewer than 20 classifications may be discarded.

In some embodiments, the subset of neurological indicative-positive classifications may be discarded because, for example, they may be random readings, of low reliability, inaccurate classification, incorrect classification, calibration, system error, disconnected electrodes, artifactual signals, system interference, or other signals, etc.

In some embodiments, the subset of neurological indicative-positive classification may be discarded to, for example, conserve memory space, improve processing speed, reduce energy usage, reduce heat of the system, reduce calculation costs, save processing power, save processing time, increase reliability, or decrease random access memory usage, etc.

In some embodiments, the greater number of neurological indicative-positive classifications in a row may be indicative of high reliability. The greater the reliability of neurological indicative-positive classifications, the more accurate determination of detecting one or more neurological conditions in a patient. In some cases, the greater reliability of neurological indicative-positive classifications may be indicative of the machine learning algorithm accuracy, quality of data (EEG signals), or health status of the EEG detecting system, etc.

In some embodiments, a particular time epoch may be classified as associated with one or more neurological conditions if the temporal data segments for a subset of the plurality of channels are classified as neurological indicative-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, a particular time epoch may be classified as associated with seizure if the temporal data segments for a subset of the plurality of channels are classified as seizure-positive. In some cases, the subset may be at least 5%, 10%, 20%, 30%, 40%, 50% or more of the plurality of channels. In some cases, the subset may be at most about 50%, 40%, 30%, 20%, 10%, 5%, or less of the plurality of channels.

In some embodiments, the classification may comprise assigning a probability value, by way of example between 0 and 1 or between 0 and 100, of a temporal segment being reflective of a subject having a neurological condition, for example, seizure, epileptiform activity, normal activity, etc. In some embodiments, the classification may comprise assigning a severity value, by way of example between 0 and 7, of a temporal segment being reflective of the severity of the neurological condition, for example, seizure, epileptiform activity, normal activity, etc. In some embodiments, the value assigned in the classification is a combined value reflecting both probability and severity.

In some embodiments, the classification may be a classification of the temporal segment into one of three or more categories with respect to a neurological condition. A temporal segment may be classified as, by way of example, neurological condition-positive, neurological condition-negative, neurological condition-like, uncertain neurological condition activity, non-neurological condition activity, artifact, etc. The neurological condition classifications may relate to, for example, seizure, epileptiform activity, etc. A temporal segment may be classified as, for example, seizure-positive, seizure-negative, seizure-like, uncertain seizure activity, epileptiform activity, non-seizure activity, artifact, etc.

The neurological condition classifications may pertain to one or more different neurological conditions. In some cases, the plurality of features may be classified into between 1 to 20 categories. In some cases, the plurality of features may be classified into between 1 to 10 categories. Individual categories may also be divided into sub-categories. For example, a neurological condition may be divided into one or more conditions that relate to, for example, seizure, epileptiform activity, etc.

In some embodiments, the method may include applying a machine learning algorithm to the plurality of features to perform a neurological multi-class classification (neurological condition-positive, neurological condition-negative, neurological condition-like, uncertain neurological condition activity, non-neurological condition activity, etc.) for each temporal segment for each channel.

In some embodiments, the method may include applying a machine learning algorithm to the plurality of features to perform a neurological multi-class classification (e.g., electrographic seizure vs. highly pathological EEG with high likelihood of epileptiform activity vs. normal electrographic activity, etc.) for each temporal segment for each channel.

In some embodiments, the method may include applying a machine learning algorithm to the plurality of features to perform a neurological binary classification (e.g., neurological condition-positive vs neurological condition-negative) for each temporal data segment for each channel. In some embodiments, the method may include applying a machine learning algorithm to the plurality of features to perform a neurological ternary classification (e.g., first neurological condition-negative vs. second neurological condition vs. first neurological condition-positive; or neurological condition-negative vs. neurological condition-indicative vs. first neurological condition-positive) for each temporal data segment for each channel. In some embodiments, the one or more features collected may be discarded prior to or during machine learning classification or prior to categorizing.

In some embodiments, the method may include applying a machine learning algorithm to the plurality of features to perform a seizure multi-class classification (seizure-positive, seizure-negative, seizure-like, uncertain seizure activity, etc.) for each temporal segment for each channel. In some embodiments, the method may include applying a machine learning algorithm to the plurality of features to perform a seizure binary classification (e.g., seizure vs non-seizure) for each temporal data segment for each channel.

In some embodiments, a human may select, and discard features prior/during machine learning classification. In some cases, a computer may select and discard features. In some cases, the features may be discarded based on a threshold value. In some cases, the features may be discarded based on a one or more corollary assessment test and/or particular values within the corollary assessment test.

In some embodiments, any number of features may be classified by the machine learning algorithm. The machine learning algorithm may classify at least 10 features. In some cases, the plurality of features may include between about 10 features to 1000 features. In some cases, the plurality of features may include between about 10 features to 200 features. In some cases, the plurality of features may include between about 10 features to 100 features. In some cases, the plurality of features may include between about 10 features to 50 features. In some embodiments, the machine learning algorithm may be, for example, an unsupervised learning algorithm, supervised learning algorithm, or a combination thereof. The unsupervised learning algorithm may be, for example, clustering, hierarchical clustering, k-means, mixture models, DBSCAN, OPTICS algorithm, anomaly detection, local outlier factor, neural networks, autoencoders, deep belief nets, Hebbian learning, generative adversarial networks, self-organizing map, expectation-maximization algorithm (EM), method of moments, blind signal separation techniques, principal component analysis, independent component analysis, non-negative matrix factorization, singular value decomposition, or a combination thereof. The supervised learning algorithm may be, for example, support vector machines, linear regression, logistic regression, linear discriminant analysis, decision trees, k-nearest neighbor algorithm, neural networks, similarity learning, or a combination thereof. In some embodiments, the machine learning algorithm may comprise a deep neural network (DNN). The deep neural network may comprise a convolutional neural network (CNN). The CNN may be, for example, U-Net, ImageNet, LeNet-5, AlexNet, ZFNet, GoogleNet, VGGNet, ResNet18 or ResNet, etc. Other neural networks may be, for example, deep feed forward neural network, recurrent neural network, LSTM (Long Short Term Memory), GRU (Gated Recurrent Unit), Auto Encoder, variational autoencoder, adversarial autoencoder, denoising auto encoder, sparse auto encoder, Boltzmann machine, RBM (Restricted BM), deep belief network, generative adversarial network (GAN), deep residual network, capsule network, or attention/transformer networks, etc.

In some embodiments, the machine learning algorithm may be, for example, a Naïve Bayes classifier, linear regression, logistic regression, decision trees, random forests, rotation forests, K nearest neighbors (KNN), clustering, support vector machines (SVM), or neural networks. In some cases, the machine learning algorithm may include ensembling algorithms such as bagging, boosting, and stacking. The machine learning algorithm may be individually applied to the plurality of features extracted for each channel, such that each channel may have a separate iteration of the machine learning algorithm or applied to the plurality of features extracted from all channels or a subset of channels at once.

In some embodiments, the method may apply one or more predictive models. In some embodiments, the method may apply one or more one predictive models per channel.

In FIG. 2, the machine learning classification module 250 may comprise any number of predictive models. In some embodiments, the random forest machine learning algorithm may be an ensemble of bagged decision trees. In some cases, the ensemble of bagged decision trees may classify each temporal data segment for each channel as (1) neurological condition-positive or (2) neurological condition-negative. In some cases, the ensemble of bagged decision trees may classify each temporal data segment for each channel as (1) seizure-positive or (2) seizure-negative. In some cases, the ensemble of bagged decision trees may classify each temporal data segment for each channel as (1) epileptiform activity-positive or (2) epileptiform activity-negative.

The ensemble may be at least about 1, 2, 3, 4, 5, 10, 20, 30, 40, 50, 60, 70, 80, 90, 100, 120, 140, 160, 180, 200, 250, 500, 1000 or more bagged decision trees. The ensemble may be at most about 1000, 500, 250, 200, 180, 160, 140, 120, 100, 90, 80, 70, 60, 50, 40, 30, 20, 10, 5, 4, 3, 2 or less bagged decision trees. The ensemble may be from about 1 to 1000, 1 to 500, 1 to 200, 1 to 100, or 1 to 10 bagged decision trees.

In some embodiments, the method may include applying a machine learning classifier to any number of channels. The method may include applying a machine learning classifier to at least about 1, 2, 3, 4, 5, 6, 7, 8, 9, 10, 15, 20, 25, 50, 100, 500, 1000 or more channels. The method may include applying a machine learning classifier to at most about 1000, 500, 100, 50, 25, 20, 15, 10, 9, 8, 7, 6, 5, 4, 3, 2 or less channels. The method may include applying a machine learning classifier from about 1 to 1000, 1 to 100, 1 to 25, or 1 to 5 channels.

In some cases, the plurality of EEG signals may be collected over a plurality of channels. The machine learning algorithm may be individually applied to the plurality of features extracted for each channel, such that each channel has a separate iteration of the machine learning algorithm or applied to the plurality of features extracted from all channels or a subset of channels at once. Each channel may have at least about 1, 2, 5, 10, 25, 50, or more predictive models applied. Each channel may have at most about 50, 25, 10, 5, 2, or fewer predictive models applied.

In some embodiments, the method may include applying a machine learning classifier to a subset of channels. The subset of channels may be at least about 1%, 5%, 10%, 20%, 30%, 40%, 50% or more of the total set of channels. The subset of channels may be at most about 50%, 40%, 30%, 20%, 10%, 5%, 1% or less of the total set of channels. The subset of channels may be from about 1% to 50%, 1% to 40%, 1% to 30%, 1% to 20%, 1% to 10%, or 1% to 5% of the total set of channels.

In some embodiments, the machine learning algorithm may have a variety of parameters. The variety of parameters may be, for example, learning rate, minibatch size, number of epochs to train for, momentum, learning weight decay, or neural network layers etc.

In some embodiments, the learning rate may be between about 0.00001 to 0.1.

In some embodiments, the minibatch size may be at between about 16 to 128.

In some embodiments, the neural network may comprise neural network layers. The neural network may have at least about 2 to 1000 or more neural network layers.

In some embodiments, the number of epochs to train for may be at least about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 500, 1000, 10000, or more.

In some embodiments, the momentum may be at least about 0.1, 0.2, 0.3, 0.4, 0.5, 0.6, 0.7, 0.8, 0.9 or more. In some embodiments, the momentum may be at most about 0.9, 0.8, 0.7, 0.6, 0.5, 0.4, 0.3, 0.2, 0.1, or less.

In some embodiments, learning weight decay may be at least about 0.00001, 0.0001, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, or more. In some embodiments, the learning weight decay may be at most about 0.1, 0.09, 0.08, 0.07, 0.06, 0.05, 0.04, 0.03, 0.02, 0.01, 0.009, 0.008, 0.007, 0.006, 0.005, 0.004, 0.003, 0.002, 0.001, 0.0001, 0.00001, or less.

In some embodiments, the machine learning algorithm may use a loss function. The loss function may be, for example, regression losses, mean absolute error, mean bias error, hinge loss, Adam optimizer and/or cross entropy.

In some embodiments, the parameters of the machine learning algorithm may be adjusted with the aid of a human and/or computer system.

In some embodiments, the machine learning algorithm may prioritize certain features. The machine learning algorithm may prioritize features that may be more relevant for detecting one or more neurological conditions, a particular neurological condition, a state of a subject associated with a neurological condition, seizure, epileptiform activity, or both. The feature may be more relevant for detecting one or more neurological conditions if the feature is classified more often than another feature. The feature may be more relevant for detecting seizure, epileptiform activity, or both if the feature is classified more often than another feature. In some cases, the features may be prioritized using a weighting system. In some cases, the features may be prioritized on probability statistics based on the frequency and/or quantity of occurrence of the feature. The machine learning algorithm may prioritize features with the aid of a human and/or computer system.

In some embodiments, one or more of the features may be used with machine learning or conventional statistical techniques to determine if a segment is likely to contain artifacts. FIG. 2 shows the artifact rejection module 255 which identifies segments containing artifacts. The identified artifacts may be a result of electrical interference, electrode instability or movement, subject movement, subject eye movement or blinking, subject chewing, subject muscle tensing, subject electrocardiographic artifact, etc. In some cases, movement sensors or other sensors may be used as an additional input to the artifact rejection module. In some cases, the identified artifacts may be rejected from being used in stroke classification. In some cases, the identified artifacts may be reduced, maycelled, or eliminated and the remaining signal may still be processed for stroke classification.

In some cases, the machine learning algorithm may prioritize certain features to reduce calculation costs, save processing power, save processing time, increase reliability, or decrease random access memory usage, etc.

### III. Neurological Condition Probability/Classification and Output

### Control Policy and Neurological Condition Probability/Classification

In some embodiments, the multi-neurological classification may include classifying each temporal data segment for each channel as (1) first neurological condition-positive, (2) second neurological condition-positive, or (3) one or both of first and second neurological condition negative. In some embodiments, the multi-neurological classification may include classifying each temporal data segment for each channel as (1) seizure-positive, (2) epileptiform activity-positive or (2) seizure-negative.

The multi-neurological classification may use predictive models as described elsewhere herein. The method may include aggregating the multi-neurological classifications for the plurality of temporal data segments for the plurality of channels over a moving time window. The method may include aggregating seizure classifications for the plurality of temporal data segments for the plurality of channels over a moving time window. The classifications may be used to determine the level of seizure or seizure risk of a patient on a particular scale or assessment test.

The method may include aggregating the multi-seizure classifications for the plurality of temporal data segments for the plurality of channels over a moving time window. The method may include aggregating the multi-seizure classifications for the plurality of temporal data segments for the plurality of channels over a moving time window. The method may include aggregating the multi-seizure classifications for the plurality of temporal data segments for the plurality of channels over a moving time window.

The aggregated neurological classifications may be subjected to a control policy module 275 of the neurological condition probability/classification calculation and output module 270, as shown in FIG. 2. FIG. 2 shows the neurological condition probability/classification calculation and output module 270. As shown in FIG. 2, the neurological condition probability/classification calculation and output module 270 may comprise a control policy module 275, a neurological condition probability/classification calculation module 280, a neurological condition probability plot module 285, and a neurological condition notification module 290. The control policy module 275 may be configured to implement a control policy, the neurological condition probability/classification calculation module 280 may be configured to calculate a neurological condition probability, the neurological condition probability plot module 285 may be configured to plot neurological condition probability values, and the neurological condition notification module 290 may be configured to provide notifications and/or assessments as described elsewhere herein, respectively.

The processing module 120 may be further configured to generate a visual output. The visual output may include a graph that displays a probability/severity that the subject is experiencing one or more neurological conditions. The graphical representation may be a combination of a plurality of different temporal graphs corresponding to the plurality of neurological conditions. The graphical representation may include an overlay of the plurality of different temporal graphs.

The processing module 120 may be configured to generate one or more corollary assessment scores that are indicative of the severity of one or more neurological conditions. The processing module 120 may be configured to generate a diagnostic output 290 based on the indications or assessments. The diagnostic output may include an aggregate wellness score or a graphical representation of the subject's brain state. The aggregate wellness score may be a combination of a plurality of discrete scores corresponding to the plurality of neurological conditions. The plurality of discrete scores may be combined based on different weights allocated to the plurality of neurological conditions. The processing module 120 may be configured to generate one or more corollary assessment scores that are indicative of one or more neurological conditions. The method may include generating one or more notifications when the patient has a wellness score below or above a particular wellness score.

In some cases, the moving window may have a period of time between 1 minute and 1 hour. In some cases, the period of time of the moving window may be dynamic or adjustable instead of fixed. In some cases, the period of time of the moving window may be dependent on the subject.

In some embodiments, a cluster of neurological condition classifications on one or more channels may be subjected to a control policy module 275 to result in an overall determination of a neurological condition for the patient for a corresponding time epoch. The control policy may be a set of rules that result in an overall determination of neurological condition diagnosis or probability for the patient. The control policy may be a set of rules that result in an overall determination of seizure diagnosis or probability for the patient. The control policy may take a set of parameters as input and act on the set of parameters according to the set of rules to result in an overall determination of a neurological condition for the patient. The control policy may take a set of parameters as input and act on the set of parameters according to the set of rules to result in an overall determination of a seizure level for the patient. The set of rules may be as described elsewhere herein. The set of rules may be adjusted at any point of time to act on more parameters or to act on less parameters. The set of rules may be adjusted at any point of time to include more rules or to remove rules. The set of rules may be at least about 1, 2, 3, 4, 5, 6 7, 8, 9, 10, 15, 20, 25, 50, 100, 500, 1000, or more rules. The set of rules may be at most about 1000, 500, 100, 50, 25, 20, 15, 10, 9, 8, 7, 6, 5, 4, 3, 2, or less rules. The set of rules may be from about 1 to 1000, 1 to 500, 1 to 100, 1 to 25, 1 to 10, 1 to 5, or 1 to 3 rules.

In some embodiments, the input of parameters for the control policy may include, the quantity of classification of channels as neurological condition-positive, the quantity of classification of channels as neurological condition-negative, the classification of channels as neurological condition-positive, the classification of channels as neurological condition-negative, the classification of channels as neurological conditions, the corresponding time epoch, the quantity of channels, the machine learning algorithm used for classification, a moving window time length, the quality of the connection of each channel, information derived from EKG signals, information derived from EMG signals, information regarding the patient's demographics, information regarding the patient's current or previous condition, information regarding treatments or medications applied to the patient, information derived from movement sensors (e.g. an accelerometer or inertial measurement unit), etc.

In some embodiments, the control policy may have any number input of parameters. The control policy may have an input of at least about 1, 2, 3, 4, 5, 6, 7, 8, 9, 10, 11, 12, 13, 14, 15, 20, 25, 50, 100, 500, 1000, or more parameters. The control policy may have an input of at most about 1000, 500, 100, 50, 25, 20, 15, 14, 13, 12, 11, 10, 9, 8, 7, 6, 5, 4, 3, 2, or less parameters. The control policy may have an input from about 1 to 1000, 1 to 500, 1 to 100, 1 to 50, 1 to 25, 1 to 15, 1 to 10, or 1 to 5 parameters.

In some embodiments, the set of rules may dictate that the control policy discards the classification of a channel. For example, if the control policy receives an input of a single a neurological condition-positive classification for a corresponding time epoch, the set of rules may discard the neurological condition-positive classification for the corresponding time epoch. For example, if the control policy receives an input of a single seizure-positive classification for a corresponding time epoch, the set of rules may discard the seizure-positive and/or classification for the corresponding time epoch.

In some cases, the control policy may receive at least about 1, 2, 3, 4, 5, 6, 7, 8, 9, 10, 50, 100, 500, or more positive classifications and the set of rules may discard each positive classification for the corresponding time epoch. In some cases, the control policy may receive at most about 500, 100, 50, 10, 9, 8, 7, 6, 5, 4, 3, 2 or less positive classifications and the set of rules may discard each stroke-positive classification for the corresponding time epoch. In some cases, the control policy may receive from about 1 to 500, 1 to 100, 1 to 50, 1 to 10, or 1 to 5 positive classifications and the set of rules may discard each stroke-positive classification for the corresponding time epoch. The positive classification may pertain to neurological conditions. The positive classifications may pertain to seizure or epileptiform activity.

In some embodiments, the set of rules may dictate that the control policy output a neurological condition-positive classification for a set of channels corresponding to a time epoch. For example, if the control policy receives a set of four or more channels that each register a neurological condition-positive classification for the corresponding time epoch, the set of rules may output a neurological condition-positive classification for the corresponding time epoch. In some cases, the control policy may receive a set of neurological condition-positive classifications of at least about 1, 2, 3, 4, 5, 6, 7, 8, 9, 10, 15, 20, 25, 50, 100, 1000, or more channels, the set of rules may output a neurological condition-positive classification for the set of stroke-positive classifications for the corresponding time epoch. In some cases, the control policy may receive a set of neurological condition-positive classifications of at most about 1000, 100, 50, 25, 20, 15, 10, 9, 8, 7, 6, 5, 4, 3, 2, or less channels, the set of rules may output a neurological condition-positive classification for the set of neurological condition-positive classifications for the corresponding time epoch. In some cases, the control policy may receive a set of neurological condition-positive classifications from about 1 to 1000, 1 to 500, 1 to 100, 1 to 50, 1 to 25, 1 to 10, or 1 to 5, the set of rules may output a neurological condition-positive classification for the set of neurological condition-positive classifications for the corresponding time epoch.

In some embodiments, the method may include calculating the neurological condition probability/classification of the patient as the percentage of neurological condition-positive classifications within a specified period of time.

As shown in FIG. 2, the neurological condition probability/classification calculation module 280 may be configured to calculate the neurological condition probability of the patient. The neurological condition probability/classification calculation module 280 may be configured to classify the neurological condition of the patient (e.g., seizure, epileptiform activity, etc.) In some cases, the period of time used for neurological condition probability/classification calculation may be between 1 minute and 1 hour. In some cases, the period of time used for neurological condition probability/classification calculation may be the entirety of the recording session. In some cases, the period of time used for neurological condition probability/classification calculation may be dynamic or adjustable instead of fixed.

In some embodiments, the neurological condition probability may form a continuous output measured by calculating neurological condition probability for a moving window of time to result in a neurological condition probability/classification calculation for individual sequential periods of time. In some cases, the period of time of the moving window may be between 1 minute and 1 hour. In some cases, the period of time of the moving window may be dynamic or adjustable instead of fixed. In some cases, the sequential periods of time formed by the moving window may be overlapping. In some cases, sequential periods of time formed by the moving window may be non-overlapping. In some cases, the moving window may move in time increments between 1 second and 1 hour. In some cases, the moving window may pause or skip periods of time such that the resulting neurological condition probability/classification calculation values are not continuous or not sequential in time.

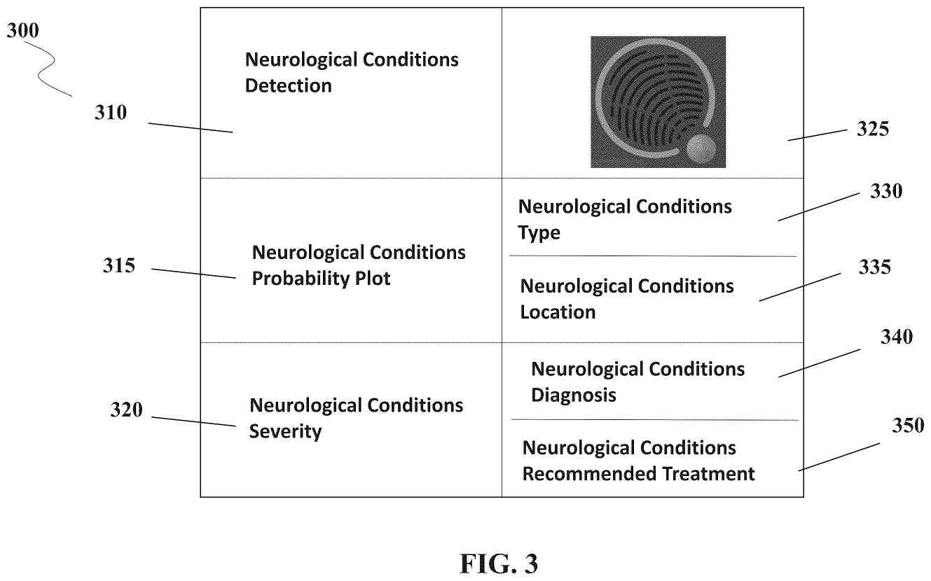

FIG. 2 shows a neurological condition probability plot module 285 configured to plot the neurological condition probability of a subject. As shown in FIG. 3, the neurological condition probability output 315 may be displayed to the user via an interface 300. The severity of the one or more neurological conditions 320 may also be displayed to the user. Additionally or alternatively, one or more of the neurological conditions type 330, neurological conditions location 335, neurological conditions diagnosis 340, and neurological conditions recommended treatment 350 may be displayed to the user. In some embodiments, the neurological condition probability output may display the one or more adjustable thresholds to the user on the time-series plot. In some embodiments, the neurological condition probability output may be displayed to the user as a time-series plot, bar graph, or chart etc.

In some embodiments, the time-series plot may be depicted in a certain color to note the threshold that has been passed or a particular severity has been reached. For example, if the probability of a seizure goes above a seizure threshold probability/severity value, a notification may be sent by the system. In some cases, the neurological condition probability plot module may display a wide variety of information, for example, the time period measured, the date, or the initial time acquisition, etc. In some cases, the neurological condition probability plot may be usable by a healthcare practitioner to assess the condition of the subject and determine a course of treatment. The neurological condition probability plot may also be usable by a healthcare practitioner to monitor the progression of the subject's condition over time or to monitor the effectiveness of courses of treatment.

FIG. 2 shows a notifications module 290 configured to generate notifications. In some embodiments, the method may include generating one or more notifications when neurological condition classifications have been made. In some embodiments, the method may include generating one or more notifications when seizure or epileptiform activity classifications have been made. In some embodiments, the method may include generating one or more notifications when neurological condition-positive classifications have been made or when the neurological condition probability is equal to or exceeds one or more thresholds. In some cases, when the neurological condition probability/classification calculation value is equal to or exceeds an adjustable threshold value the system may display to a subject (e.g., patient) or user (e.g., healthcare practitioner, doctor, nurse, etc) a notification that the system has detected continuous neurological condition activity. The notification may also include any color. For example, the background of the screen displaying the notification may be red. The text of the notification may be any color, for example, white. The color of the background of the screen may correlate with the value of the neurological condition probability calculation. For example, if the neurological condition probability is equal to or above a certain threshold, the selected color for the background of the screen may indicate that the neurological condition probability is equal to or above a threshold. The color of the text of the notification may correlate with the value of the neurological condition probability calculation. For example, if the neurological condition probability calculation is equal to or above a certain threshold, the selected color for the text of the notification may indicate that the stroke probability is equal to or above a threshold.

In some embodiments, the method may include generating one or more notifications when seizure—, epileptiform activity—, or any combination thereof—positive classifications have been made or when the seizure—, epileptiform activity—or a combination thereof probability is equal to or exceeds one or more thresholds. In some cases, when the seizure—, epileptiform activity—or a combination thereof probability/classification calculation value is equal to or exceeds an adjustable threshold value the system may display to a subject (e.g., patient) or user (e.g., healthcare practitioner, doctor, nurse, etc.) a notification that the system has detected continuous seizure-, epileptiform activity—, or a combination thereof activity.

The system may also display a wide variety of information to the subject or user in addition to the notification of detected continuous neurological condition activity. The system may display the neurological condition probability plot 315, severity of a neurological condition 320, location of the neurological condition within a subject 335, likelihood of a neurological condition to occur, classification of the neurological condition type 330, the time period for which the continuous neurological condition activity was detected (e.g., 7:40 pm to 7:50 pm), etc. For example, the location of the neurological condition within the subject 335 may be a region of the subject's brain where an abnormality associated with seizure or epileptiform activity was recorded. As another example, the location of the neurological condition within the subject 335 may be one or more channels or electrodes where an abnormality associated with seizure or epileptiform activity was observed. The one or more notifications may be usable by a healthcare practitioner to assess the condition of the subject and determine a course of treatment. The notification may provide a diagnosis output to the healthcare practitioner. In some embodiments, the diagnosis output may include a neurological condition classification that is selected from a plurality of different neurological condition classes or neurological condition types. In some cases, the method may provide one or more neurological condition classifications described elsewhere herein. In some cases, the diagnosis output may provide symptoms pertaining to a neurological condition classification that correlate to a particular set of EEG signals. In some embodiments, the information in the diagnosis output may be useable to identify or detect one or more neurological condition pathologies.

In some embodiments, one or more notifications (e.g., diagnosis output) may be generated when the neurological condition probability value is equal to or exceeds one or more thresholds as described elsewhere herein. In some embodiments, one or more notifications (e.g., diagnosis output) may be generated when the neurological condition classification of the one or more features has been completed. In some cases, the one or more notifications may be generated in the form of visual, audio, and/or textual alerts. The device may include speakers 325 to provide audio notifications. In some cases, the one or more notifications may be delivered via networked communication technology such as the internet, telephone, facsimile, pager, short message service, etc. In some cases, the form, content, or delivery mechanism of the one or more notifications generated may depend on the neurological condition probability value. In some cases, the user may be able to select the form, content, or delivery mechanism of the one or more notifications generated.

In some embodiments, the neurological condition detection output may include an interface. The interface may provide indication of the EEG signal activity for the plurality of channels from the data module 110. The interface may display parameters that a user may adjust, for example, the time display, the scale, the high pass frequency, the low pass frequency, or the notch value, etc. The interface may also provide a neurological condition probability plot as described elsewhere herein. The interface may also provide neurological condition probability burden results over different time periods. The interface may also depict the stroke probability determination for each time segment. The interface may also provide a mechanism for the user to accept or reject the algorithm derived neurological condition probability determination or neurological condition classification. The interface may also provide a mechanism for the user to input their own determination of a neurological condition containing segments or neurological condition classification. In some cases, the neurological condition probability and/or neurological condition classification may be adjusted as a result of user entered information regarding neurological condition episodes. The displayed neurological condition probability/neurological condition classification and neurological condition notifications may be based solely on algorithm derived neurological condition determination, solely on user entered neurological condition determination, or on a combination of algorithm and user neurological condition determination.

In some embodiments, the neurological condition probability/classification calculation module may calculate neurological condition probability. The neurological condition notification module may output a notification if the neurological condition probability value crosses a threshold value. In some cases, notifications may be generated to a specific person that the method is programmed to notify. The threshold for notification may also be user adjustable.

In some embodiments, the neurological condition probability/classification calculation and output module may utilize criteria in addition to the neurological condition probability threshold to output a notification. In some cases, dynamic criteria may be applied with a combination of time based, neurological condition probability based, and other policies to determine if a notification is output.

In some embodiments, the neurological condition probability/classification calculation module may provide a probability value of a neurological condition to occur. The probability value may be provided as a percentage. The probability value may be provided as scaled-values (e.g., 1 to 10, 0 to 100, etc.). The probability values may be indicative of confidence of a neurological condition and/or the severity of the neurological condition. The probability value may be provided via a notification or a time-series plot as described elsewhere herein.

In some embodiments, the data may further include non-EEG data, wherein the non-EEG data comprises blood pressure, heart rate or motion data of the subject. In some cases, the EEG data and the non-EEG data are processed in a complementary or synergistic configuration to generate the diagnosis output or improve an accuracy of the diagnosis output. In some cases, the method is performed with the data collected from the subject in an external environment outside of a standard healthcare facility. In some cases, the external environment includes a pre-hospital location, a field environment or an ambulatory setting.

In some embodiments, the system may be coupled with other systems. In some cases, the systems may be eye trackers, movement sensors (e.g., an accelerometer or an inertial measurement unit), electromyography (EMG), electrocardiogram (ECG or EKG), etc. The collection of EEG data may be complemented with other inputs including, observed symptoms, other commonly collected biological inputs, such as heart signals (ECG or other heart rate monitors) and blood pressure, and passively collected movement measurements, such as from accelerometers and gyroscopes (for changes in movement, blood flow, and artifact detection), or questionnaire inputs collected from the user. Collection of the EEG and complementary input data may be rapidly used in an ambulance or other pre-hospital location, by practitioners in a hospital for quick triage in an emergency department (ED), for longer-term patient monitoring in an intensive care unit (ICU), and also perioperatively, before, during, and after surgical or other in-hospital procedures. For such procedures, separate or continuous recordings may be done in order to monitor for changes in patient health such as brain degradation from any neurological or cardiovascular complications (edema, swelling, etc.), and may also allow for more individualized, patient-based algorithm features, processing, tracking and baseline comparisons with baseline patient data (including but not limited to focal slowing, asymmetries, changes in delta and theta to delta ratios).

The complimentary non-EEG sensor data may be used for data selection or as independent features in the machine learning algorithm. The non-EEG data may be used to complement the EEG features to improve performance of the algorithm.

In some embodiments, the method may include transmitting the diagnostic output over one or more wired or wireless networks substantially in real-time to enable remote stroke management and care for the subject.

In some embodiments, the diagnostic output comprises a single diagnosis, a binary diagnosis, or a multi-tiered diagnosis associated with the neurological condition or the onset of the one or more neurological condition.

In some embodiments, the method may further be extendable and configured for diagnosis of acute traumatic brain injuries. In some cases, the EEG signals may include signal patterns that are associated with, or indicative of asymmetries in different areas and hemispheres of the subject's brain. In some embodiments, the EEG signals are passively collected using a set of electrodes worn on the subject's head. In some cases, the plurality of features includes at least fifty distinct features. In some cases, the plurality of features includes at least one hundred distinct features.

### IV. Seizure Classification

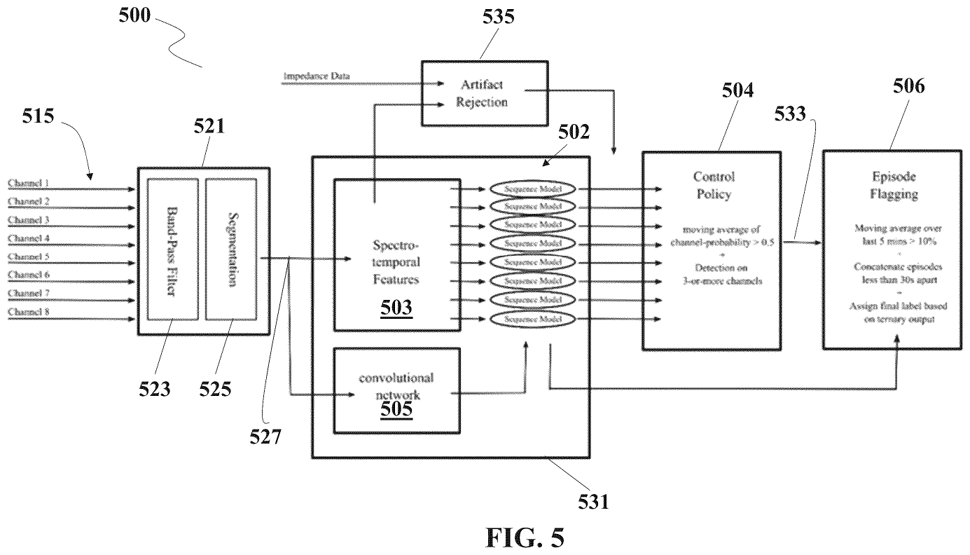

FIG. 5 schematically shows an exemplary seizure classification module 500 for analyzing acquired sections of EEG recordings from channels 515. The seizure classification module may be used to assist health service providers in the assessment of seizure using a prediction module (including, e.g., a predictive or machine learning model) 531, a control policy module 504 and an episode flagging module 506. The EEG recordings may be obtained from a plurality of electrodes that may be coupled to or incorporated into a headband, headgear, or other apparatus configured to place the electrodes on or around the head of a subject. For example, the EEG recordings may be obtained from between 2 and 20 electrodes coupled to or incorporated into a headband (e.g., 8, 10, or 16 electrodes coupled to the headband). The seizure classification module 500 may include a preprocessing module 521 configured to preprocess incoming EEG recordings. The preprocessing module 521 may include a filtering module 523 and a segmentation module 525 to process each of EEG recording from channels 515. For example, EEG recordings may be sampled at a rate of between about 50 Hz and about 350 Hz and band-pass filtered between about 0.5 Hz and about 40 Hz by filtering module 523. For example, the EEG recordings may be sampled at a rate of about 250 Hz and band-pass filtered between about 1 Hz and about 35 Hz using a 5th order Butterworth filter. Filtered EEG signals may be divided by segmentation module 525 to generate preprocessed temporal segments 527. Each temporal segment may have a duration of between about 1 and about 120 seconds, such as about 10 seconds. In some variations, the temporal segments may be non-overlapping. In some variations, filtered EEG signals may be divided into shorter temporal segments (e.g., segments of about 6 seconds) with a period of overlap between consecutive segments (e.g., 1 second overlap) to provide finer temporal resolution for the signals.

The temporal segments 527 may be further processed by prediction module 531. The prediction module 531 may include one or both of a single-channel feature extraction module 503 and a multi-channel feature extraction module 505. Single-channel feature extraction module 503 may be configured to extract a predetermined set of features from each of the temporal segments 527. For example, single-channel feature extraction module 503 may extract a plurality of different features, including one or more time-domain features and one or more frequency-domain features. For example, features may include systemic and random variability in power in a frequency band over time. Below, Table 1 lists and briefly describes nonlimiting examples of features which may be computed by single-channel feature extraction module 503. Nonlimiting examples of time-domain features which may be extracted by feature extraction module 503 include: amplitude range, RMS of the amplitude, standard deviation of the amplitude, sharpness, area under the wave, number of local minima and/or maxima, peak amplitude, zero-crossings, RMS of the derivative of the signal, and regularity. Examples of frequency-domain features extracted by feature extraction module 503 include but are not limited to: dominant frequency, dominant frequency power, leakage of signal outside of the dominant frequency, spectral entropy, power of signal in a given frequency band (e.g., alpha band, beta band, gamma band, delta band, or theta band). The plurality of different features that may be extracted may also include power in different frequency bands (for example, alpha, beta, delta, theta, and gamma), spectral properties, power ratios, amplitude characteristics and morphology features, entropy, variability and wavelet decomposition.

TABLE 1

| Feature Name | Brief Description |
|---|---|
| Activity | Hjorth Activity |
| ArtD_RMS | Root Mean Square (Sliding Window) |
| AuxCount1 | Bandpass and auxiliary counts algorithm |
| AuxCount2 | |
| AuxCount3 | |
| Complexity | Hjorth Complexity |
| DF | Dominant Frequency |
| DFPower | Power at the dominant frequency |
| Entropy | Entropy |
| Kurt | Kurtosis |
| Leak | Leakage of the signal outside of dominant |
| | frequency |
| Mobility | Hjorth Mobility |
| Range | Amplitude range |
| Sharpness | Measure of sharpness of the EEG signal |
| Skew | Skewness |
| Area | Area under the wave |
| DCT_1D | 1D Discrete Cosine Transformation |
| EdgeFreq | Spectral edge frequency below which 90% of |
| | the power of the signal lies |
| Energy | Signal energy |
| Entropy2 | Entropy |
| lineLen | Line length |
| localMinMax | Number of local Min/Max |
| normDecay | Normalized decay |
| PeakAmp | Peak Amplitude |
| PosZeroCross | Number of positive zero crossings of the signal |
| RMS | RMS of the entire segment |
| SpectralEnt | Spectral Entropy |
| ValAmp | Valley Amplitude |
| Alpha_Delta_Ratio_{—} | Alpha/Delta power ratio |
| Modified | |
| Smooth_{—} | Delta/Alpha power ratio with numeric |
| DeltaAlphaRatio | correction |
| Smooth_{—} | Theta/Alpha power ratio with numeric |
| ThetaAlphaRatio | correction |
| AV_STD | Standard deviation of EEG amplitudes |
| Max_Jump | Maximum point to point jump in the signal |
| RMS_of_DIFF | RMS of derivative of the signal |
| LFMax | Detects the maximum negative to positive zero |
| | crossing frequency |
| NLEO | Nonlinear energy operator |
| ApEn_Modified | Approximate Entropy, another measure of |
| | entropy of the signal |
| PMRS_modified_{—} | Quantifies the regularity of a signal |
| ver1_AT | |
| Hurst | Estimate of the fractal index H of the input signal |
| Delta | Power in Delta band (0.5-4 Hz) |
| Theta | Power in Theta band (3-7 Hz) |
| Alpha | Power in Alpha band (7-12 Hz) |
| Beta | Power in Beta band (13-25 Hz) |
| GammaB | Power in Gamma Band (25-125 Hz) |
| Delta_N | Power in Delta band (0.5-4 Hz) normalized by |
| | total power |
| Theta_N | Power in Theta band (3-7 Hz) normalized by |
| | total power |
| Alpha_N | Power in Alpha band (7-12 Hz) normalized by |
| | total power |
| Beta_N | Power in Beta band (13-25 Hz) normalized by |
| | total power |
| GammaB_N | Power in Gamma Band (25-125 Hz) |
| | normalized by total power |
| TotalPower | Total power for frequencies under the Nyquist |
| | frequency |
| Temporal | R² loss when predicting current EEG using past |
| predictability | data |
| Spatial | R² loss when predicting EEG using other |
| Predictability | channels |
| ACF Height | Height of the second auto-correlation peak |
| ACF Width | Temporal location of second auto-correlation |
| | peak |
| ACF Area | Area under the absolute value of auto-correlation |
| | function |
| Correlation F | Correlation coefficient between an electrode and |
| | all others in the same hemisphere |

Multichannel feature extraction module 505 may be configured to extract a predetermined set of multi-channel features that quantify a degree of correlation between pairs of segments from different EEG signals corresponding to a given time epoch. Unlike single channel features that characterize a given segment from an EEG signal received from one channel, multichannel features characterize inter-channel interactions within a given time epoch. Nonlimiting examples of multichannel features which may be extracted include: an average correlation coefficient for the EEG signal waveform received from pairs of electrodes within and/or across hemispheres (the waveforms may be filtered to isolate signals within alpha, beta, gamma, delta, or theta frequency bands), an average peak correlation (measured over different lags) within and/or across hemispheres, an average lag at which peak correlation is observed within and/or across hemispheres, and an average correlation coefficient of a power spectrum within and/or across hemispheres. In some variations, the multi-channel features that may be computed to quantify inter-channel interactions may include correlation within and across hemispheres for different frequency bands (for example, alpha, beta, delta, theta, and gamma), as well as spectral, amplitude and phase related correlations and synchrony measures. Multichannel feature extraction module 505 may be a neural network (e.g., a multi-layer, multi-channel cascaded convolutional neural network) trained on filtered EEG to learn data-driven features that are the best markers for the EEG categories of interest. A training process for multichannel feature extraction module 505 may include one or more deep learning techniques such as data augmentation, batch-normalization, max-pooling, skip-connections, transfer learning, 1D and 2D convolution, and early stopping to facilitate the feature recognition process.

Each temporal segment may also be evaluated by artifact rejection module 535. A combination of some of the features extracted by single-channel feature extraction module 503 for each of the segments may be used by artifact rejection module 535 to determine if a given temporal segment comprises an artifactual signal or an excess of artifacts in the signal and should be excluded from further analysis to contribute to the final seizure classification. Artifact rejection module 535 may also use an impedance measurement of each electrode used to record EEG signals to determine whether to exclude the segment from further analysis. For example, if the most recently detected impedance on an electrode is higher than a predetermined threshold, the temporal segment may be marked as an artifact and may not be used for subsequent analysis/predictions.

Prediction module 531 may include sequence models 502 for generating seizure classifications. For example, prediction module 531 may include 8 sequence models corresponding to 8 EEG channels. Sequence models 502 may look backward in time to learn temporal associations and evolutions of EEG signals. For example, one or more sequence model may look over a 5-minute window of a pre-recorded and preprocessed EEG signal to classify a current EEG segment (e.g., a current 10 second segment). Additionally or alternatively, sequence models 502 may look forward in time learn temporal associations and evolutions of EEG signals. The sequence models 502 may be trained on a combination of pre-defined features and data-driven, cascaded convolutional filters. In some variations, random under-sampling on the individual channel features may be employed during training. Random under-sampling of the data (e.g., 80% of the minority class available for training) may allow selection of a subset of the data such that the number of seizure classifications (e.g., ternary labels including “electrographic seizure”, “highly pathological electrographic activity”, and “normal electrographic activity” class labels) visible to the model for training may be equalized. The sequence models 502 may predict whether an EEG signal recorded from a subject is anomalous by generating a class label for an EEG segment for one or more input signals. For example, sequence models 502 may predict the likelihood (e.g., a probability between 0 and 1 or between 0 and 100) of an EEG segment (e.g., a 10 second temporal segment) under two scenarios: (1) Normal EEG vs. Highly Pathological EEG, and (2) Seizure vs. Not Seizure. The EEG segment may be evaluated under the first scenario (Normal EEG vs. Highly Pathological EEG) before, after, or at the same time as it is evaluated under the second scenario (Seizure vs. Not Seizure). The respective outputs of the two scenarios may be combined to label a class for each segment-Class 1 (electrographic seizure-like EEG), Class 2 (highly pathological EEG with high likelihood of epileptiform activity), and Class 3 (normal EEG).

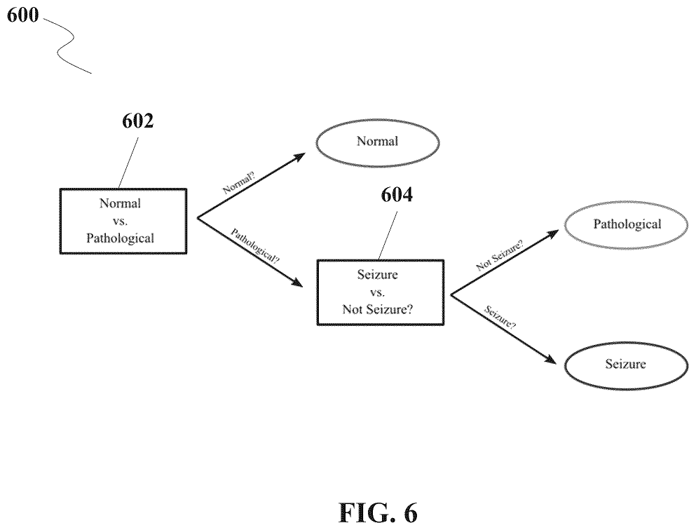

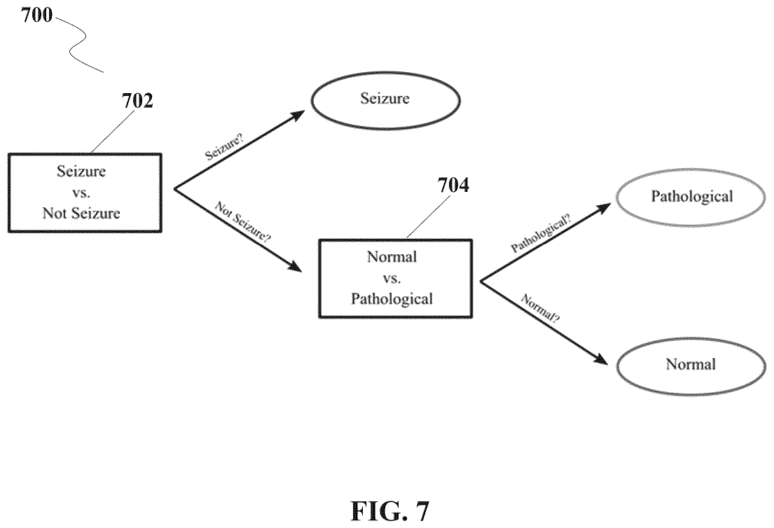

Sequence models 502 may output ternary Class 1, Class 2, or Class 3 seizure labels for one or more EEG segments by the logic sequence 600 shown schematically in FIG. 6. The first step 602 of logic sequence 600 may be predicting the likelihood of a segmented EEG segment under the Normal EEG vs. Pathological EEG scenario. If the probability of the segment as normal electrographic activity outweighs the probability of the segment as epileptiform activity, the segment may be classified as Class 3. Alternatively, if the probability of the segment as epileptiform activity outweighs the probability of the segment as normal electrographic activity, the segment may be classified as Class 2. When step 602 results in labeling a segment as Class 2, the second step 604 of logic sequence 600 may be predicting the likelihood of an EEG segment under the Seizure vs. Not Seizure (i.e., Pathological) EEG scenario. Similarly, the segment may be labeled Class 1 or Class 2 by comparing the probabilities of the EEG segment. Similarly, sequence models 502 may output such ternary labels by the logic sequence 700 shown schematically in FIG. 7. The first step 702 of logic sequence 700 may be predicting the likelihood of a segmented EEG segment under the Seizure vs. Not Seizure scenario. If the probability of the segment as electrographic seizure-like activity outweighs the probability of the segment as epileptiform activity, the segment may be classified as Class 1. Alternatively, if the probability of the segment as epileptiform activity outweighs the probability of the segment as electrographic seizure-like activity, the segment may be classified as Class 2. When step 602 results in labeling a segment as Class 2, the second step 604 of logic sequence 600 may be predicting the likelihood of an EEG segment under the Normal EEG vs. Pathological EEG scenario. Similarly, the segment may be labeled Class 2 or Class 3 by comparing the probabilities of the EEG segment.

In some variations, the prediction module 531 may further subdivide the three primary seizure classifications (e.g., “electrographic seizure”, “highly pathological electrographic activity”, and “normal electrographic activity”) and provide the subclassifications for clinical use. For example, “highly pathological electrographic activity” may be subdivided into “generalized” or “focal” pathology depending on the spatial characteristics of the processed signal output. As another example, “normal electrographic activity” may be subcategorized into “abnormal background signal” (marked by the presence of slowing EEG signal) vs. “normal background signal”.

In some variations, the prediction module 531 may additionally or alternatively provide a seizure severity value associated with an EEG segment. For example, the prediction module 531 may provide a seizure severity value and a seizure probability for one or more segments. The seizure severity value of an EEG segment may be a value that is equal to or between about 0 and any greater value, or a value equal to or between about 1 and any greater value. For example, the seizure severity value may be equal to or between 0 and 1, equal to or between 0 and 10, or equal to or between 0 and 100 (e.g., between 0 and 7). As another example, the seizure severity value may be equal to or between 1 and 10 or equal to or between 1 and 100. The low threshold value (e.g., 0 or 1) may indicate no seizure (normal EEG), and the high threshold value (e.g., 1, 10, or 100) may indicate an electrographic seizure. Seizure severity values between the low and high threshold values may indicate highly pathological electrographic activity in a subject and/or may indicate subclassifications of seizure beyond ternary class labels (e.g., “highly pathological electrographic activity-generalized pathology”, “highly pathological electrographic activity-focal pathology”, “normal electrographic activity-abnormal background signal”, “normal electrographic activity-normal background signal”).

The seizure classifications determined by prediction module 531 (e.g., Class 1, Class 2, and Class 3 labels for each segment) may be transmitted to other modules, devices, or components of a system. In some variations, the classifications generated by prediction module 531 may be preliminary class labels that may be further processed (e.g., binarized) by one or more other modules. For example, one or more outputs of the prediction module 531 (e.g., the ternary seizure-activity class labels based on the probability output for the Normal EEG vs. Pathological EEG classifier, and in some variations the probability output for the Seizure vs. Not Seizure classifier) may be combined over a time epoch and processed against a first pre-defined set of rules, schematically shown as control policy module 504, as a first step in determining an overall seizure classification for a subject. For example, the control policy module 504 may calculate a moving average of an output of the prediction module 531, such as the probability output for the Normal vs. Pathological EEG classifier or the probability for the Seizure vs. Not Seizure classifier, over the time epoch. The time epoch may include one or more EEG segments, such as between about 1 and about 10 segments (e.g., about 3 continuous temporal segments). If the calculated moving average is below a predetermined threshold, then at least a portion of the combined output may retain its class label or may be relabeled with a new class label. Additionally or alternatively, if the combined output is the result of an input EEG signal recorded on fewer than a predetermined number of channels, then at least a portion of the combined output may retain its class label or may be relabeled with a new class label. For example, the control policy module 504 may calculate a moving average of the Normal EEG vs. Pathological EEG probability over a 30 second time epoch including a combination of three 10 second continuous temporal segments. If the moving average is below a predetermined threshold, such as below about 0.5, or if the 30 second time epoch resulted from an EEG signal recorded on fewer than a predetermined number of channels, such as fewer than about 3 channels, then the middle temporal segment within the time epoch may be reset to a Class 3 (“normal EEG”) label. Alternatively, if the moving average is above the predetermined threshold and the 30 second time epoch resulted from an EEG signal recorded on more than the predetermined number of channels, then the middle temporal segment within the time epoch may retain its original class label from the prediction module 531 (i.e., either retain a Class 3 “normal EEG” label or a Class 2 “highly pathological EEG” label).

In some variations, an overall seizure classification generated by control policy module 504 may be transmitted to episode flagging module 506. That is, one or more outputs of the control policy module 504 (e.g., a binarized Class 3 “normal EEG” or Class 2 “highly pathological EEG” label corresponding to an epoch or a segment) may be combined over a time window and processed against a second pre-defined set of rules, schematically shown as episode flagging module 506, as a second step in determining the overall seizure classification for a subject. For example, the episode flagging module 506 may calculate a moving average of one or more outputs 533 of the control policy module 504, such as the binarized probability output for the Normal EEG vs. Pathological EEG classifier or the Seizure vs. Not Seizure classifier, over the time window. The time window may encompass one or multiple successive time epochs, such as between about 1 and about 20 time epochs (e.g., about ten 30 second time epochs). In some variations, successive epochs that are separated by less than a predetermined period may be merged into a single epoch prior to further processing by one or both of control policy module 504 and episode flagging module 506. For example, continuous epochs that are separated by less than 30 seconds may be merged into a single epoch prior to further processing. If a prevalence of one of the binarized classifications (e.g., Class 2 “highly pathological EEG” or Class 3 “normal EEG”) is above a predetermined threshold over the EEG time window, at least a portion of the time window may be labeled with an overall seizure classification of the same label. For example, if the prevalence of the Class 2 label is over between about 5% and about 50%, such as over about 10%, over a 5-minute time window (i.e., an EEG window including ten 30 second time epochs), then each time epoch within the time window that is associated with a Class 2 label (i.e., each time epoch output from control policy module 504 having a middle temporal segment labeled as Class 2) may receive an overall seizure activity classification of Class 2 (“highly pathological EEG”) via episode flagging module 506.

For each epoch computed and analyzed under one or both of control policy module 504 episode flagging module 506, if an associated ternary output for any of the constitute segments (e.g., temporal segments) is “Class 1” (electrographic seizure-like EEG), the epoch may receive an overall seizure activity classification of “Class 1” via episode flagging module 506.

### V. Post Neurological Condition Classification

In some embodiments, the method may include generating one or more notifications as described elsewhere herein. For example, the method may include generating one or more notifications of a location of a neurological condition within a subject. For example, one or more notifications may be indicative of a region of the subject's brain where an abnormality associated with seizure or epileptiform activity was recorded. As another example, one or more notifications may be indicative of one or more channels or electrodes where an abnormality associated with seizure or epileptiform activity was observed.

In some embodiments, the method may provide a user a response (e.g., a recommendation or instruction) to treat or prevent the detected neurological condition over a short term (e.g., in real-time or over a minute, an hour, a day, or multiple days) or over a long-term (e.g., over a week, a month, a year, or multiple years). The method may provide a response to minimize or reduce the risk of the onset of the neurological condition. In some cases, a therapeutic may be delivered to the subject to prevent and/or mitigate the predicted neurological condition. In some cases, the method may adjust the quantity of therapeutic delivered to the subject.

### VI. Computer Systems

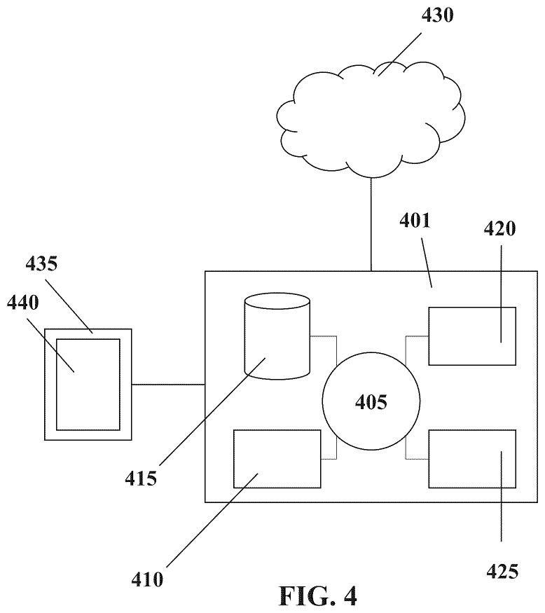

The present disclosure provides computer systems that are programmed to implement methods of the disclosure, including the control of the multi-detection system, control hardware components, receive and process data, interface with a user, etc. FIG. 4 shows a computer system 401 that is programmed or otherwise configured to operate and/or control the data module and the processing module. The computer system 401 may regulate various aspects of the present disclosure, such as, for example, determining neurological conditions, classification of neurological conditions, classification of EEG signals, classification of seizures, generate notifications, generate probability plots of neurological conditions, processing EEG signals, segmenting EEG signals, extracting features, processing features with prediction modules (e.g., predictive models), relating features to assessment scales, implementing the control policy and neurological condition burden, calculating the value, plotting the probability of the neurological conditions, etc. The computer system 401 may be an electronic device of a user or a computer system that is remotely located with respect to the electronic device. The electronic device may be a mobile electronic device.

The computer system 401 includes a central processing unit (CPU, also “processor” and “computer processor” herein) 405, which may be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 may be a data storage unit (or data repository) for storing data. The computer system 401 may be operatively coupled to a computer network (“network”) 430 with the aid of the communication interface 420. The communication interface may be wired or wireless. The network 430 may be the Internet, an internet and/or extranet, or an intranet and/or extranet that is in communication with the Internet. The network 430 in some cases is a telecommunication and/or data network. The network 430 may include one or more computer servers, which may enable distributed computing, such as cloud computing. The network 430, in some cases with the aid of the computer system 401, may implement a peer-to-peer network, which may enable devices coupled to the computer system 401 to behave as a client or a server.

The CPU 405 may execute a sequence of machine-readable instructions, which may be embodied in a program or software. The instructions may be stored in a memory location, such as the memory 410. The instructions may be directed to the CPU 405, which may subsequently program or otherwise configure the CPU 405 to implement methods of the present disclosure. Examples of operations performed by the CPU 405 may include fetch, decode, execute, and writeback.

The CPU 405 may be part of a circuit, such as an integrated circuit. One or more other components of the system 401 may be included in the circuit. In some cases, the circuit is an application specific integrated circuit (ASIC).

The storage unit 415 may store files, such as drivers, libraries and saved programs. The storage unit 415 may store user data, e.g., user preferences and user programs. The computer system 401 in some cases may include one or more additional data storage units that are external to the computer system 401, such as located on a remote server that is in communication with the computer system 401 through an intranet or the Internet.

The computer system 401 may communicate with one or more remote computer systems through the network 430. For instance, the computer system 401 may communicate with a remote computer system of a user (e.g., neurological condition detection system manager, neurological condition detection system user, neurological condition data acquirer, neurological condition detection system scribe). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user may access the computer system 401 via the network 430.

Methods as described herein may be implemented by way of machine (e.g., computer processor) executable code stored on an electronic storage location of the computer system 401, such as, for example, on the memory 410 or electronic storage unit 415. The machine executable or machine readable code may be provided in the form of software. During use, the code may be executed by the processor 405. In some cases, the code may be retrieved from the storage unit 415 and stored on the memory 410 for ready access by the processor 405. In some situations, the electronic storage unit 415 may be precluded, and machine-executable instructions are stored on memory 410.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 401, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 may include or be in communication with an electronic display 435 that comprises a user interface (UI) 440 for providing, for example, a login screen for an administrator to access software programmed to control the multi-indication detection system and functionality and/or for providing the operation status health of the multi-indication detection system. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 405. The algorithm may, for example, be component of software described elsewhere herein and may modulate the seizure detection system parameters (e.g., processing EEG signals, predictive models, control policy, neurological condition burden, notifications, etc.).

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A method for classifying seizures comprising:
obtaining data comprising electroencephalography (EEG) signals recorded from a subject over a plurality of channels;
pre-processing the data by:
dividing the data into a plurality of temporal segments, and
extracting a plurality of features from each of the temporal segments;
generating a preliminary seizure classification for each of the temporal segments based on the plurality of extracted features; and
determining an overall seizure classification for the subject based on the preliminary seizure classification for each of the temporal segments.

2. The method of claim 1, wherein the overall seizure classification is a ternary classification of electrographic seizure-like activity, highly pathological EEG with high likelihood of epileptiform activity, or normal electrographic activity.

3. The method of claim 1, wherein the overall seizure classification is a seizure probability or a seizure severity value.

4. The method of claim 1, wherein generating the preliminary seizure classification comprises generating a first probability of seizure under a first comparison and generating a second probability of seizure under a second comparison.

5. The method of claim 4, wherein generating the first probability of seizure comprises comparing a first likelihood of a temporal segment as normal electrographic activity to a second likelihood of the temporal segment as highly pathological EEG with high likelihood of epileptiform activity.

6. The method of claim 5, wherein determining the overall seizure classification comprises calculating a moving average of the first probability of seizure over the time window.

7. The method of claim 4, wherein generating the second probability of seizure comprises comparing a first likelihood of a temporal segment as electrographic seizure to a second likelihood of the temporal segment as highly pathological EEG with high likelihood of epileptiform activity.

8. The method of claim 4, wherein generating the first and second probabilities of seizures comprises filtering the each of the temporal segments with cascaded convolutional filters.

9. The method of claim 4, wherein generating the preliminary seizure classification comprises combining the first and second seizure probabilities and classifying a corresponding temporal segment based on the combined seizure probabilities.

10.-11. (canceled)

12. The method of claim 1, wherein determining the overall seizure classification comprises determining the overall seizure classification for a time window.

13. (canceled)

14. The method of claim 12, wherein determining the overall seizure classification comprises calculating a moving average of the preliminary seizure classification over the time window.

15. The method of claim 12, wherein each temporal segment corresponds to at least one epoch, and wherein the time window comprises one or more epochs.

16. The method of claim 15, wherein pre-processing the data further comprises extracting a plurality of multi-channel features that quantify a degree of correlation between pairs of temporal segments from different EEG signals corresponding to a given time epoch.

17. The method of claim 15, further comprising using a multichannel machine learning model to generate a multi-channel seizure classification for each time epoch based on the plurality of multi-channel features.

18.-23. (canceled)

24. The method of claim 1 further comprising providing a trace of the overall seizure classification over time.

25. The method of claim 24 further comprising determining a trendline of the trace.

26.-29. (canceled)

30. The method of claim 1, wherein the EEG signals are recorded from a plurality of electrodes incorporated into a headband worn by the subject.

31. (canceled)

32. The method of claim 1, further comprising treating the subject for seizure if one or both of electrographic seizure and highly pathological EEG with high likelihood of epileptiform activity and is detected.

33. A system for detecting seizure comprising:
a data module configured to receive data comprising a plurality of electroencephalography (EEG) signals recorded during a time window and over a plurality of channels from a subject; and
a seizure detection module comprising a memory storing a set of instructions and one or more processors that are configured to, responsive to the set of instructions:
pre-process the data received by the data module by:
dividing the EEG signal into a plurality of temporal segments, and
extracting a plurality of features from each of the plurality of temporal segments;
generating a preliminary seizure classification for each of the temporal segments based on the plurality of extracted features; and
determining an overall seizure classification for the subject based on the preliminary seizure classification for each of the temporal segments.

34. The system of claim 33, wherein the overall seizure classification is a ternary classification of electrographic seizure-like activity, highly pathological EEG with high likelihood of epileptiform activity, or normal electrographic activity.

35.-39. (canceled)

40. The system of claim 33, further comprising a headband, the headband comprising a plurality of electrodes from which the plurality of electroencephalography (EEG) signals is recorded.

41.-55. (canceled)
